(19) United States
(12) Patent Application Publication
(10) Pub. No.: US 2014/0142052 A1
(43) Pub. Date: May 22, 2014

(54) PHOSPHORYLATED POLYOLS, PYROPHOSPHATES, AND DERIVATIVES THEREOF HAVING BIOLOGICAL ACTIVITY

(71) Applicants: NormOxys, Inc., Wellesley, MA (US); Universite de Strasbourg, Strasbourg (FR)

(72) Inventors: Jean-Marie Lehn, Strasbourg (FR); Yves Claude Nicolau, Newton, MA (US); Srinivasu Pothukanuri, Strasbourg (FR); Adam Brockman, Arlington, MA (US); John Hey, Nashua, NH (US); Jeremy Yu, Strasbourg (FR)

(73) Assignee: NormOxys, Inc., Wellesley, MA (US)

(21) Appl. No.: 13/897,113

(22) Filed: May 17, 2013

Related U.S. Application Data
(60) Provisional application No. 61/648,222, filed on May 17, 2012.

(51) Int. Cl. C07F 9/6564; A61K 31/6615; A61K 31/7028; A61K 45/06

(52) U.S. Cl. CPC C07F 9/6564; A61K 45/06; A61K 31/6615; A61K 31/7028
USPC 514/25; 514/105; 558/86; 536/17.1

(57) ABSTRACT

The present invention provides phosphorylated and pyrophosphate derivatives of polyols, and structural derivatives of these compounds, and provides pharmaceutical compositions comprising the same. The compounds and compositions disclosed herein have various biological activities, including for example, as allosteric effectors of hemoglobin and/or as kinase inhibitors. The present invention further provides methods for therapy in human or mammalian patients, and methods for synthesis of biologically active compounds and their intermediates.

### CROSS REFERENCE TO RELATED APPLICATIONS

The present application is a nonprovisional of and claims priority to U.S. Provisional Patent Application No. 61/648,222, filed May 17, 2012, the entire contents of which is incorporated herein by reference.

### FIELD OF THE INVENTION

The present invention provides, among other things, phosphorylated and pyrophosphate derivatives of polyols, as well as structural derivatives of these compounds, for pharmaceutical use.

### BACKGROUND

Acyclic and cyclic phosphorylated/polyphosphorylated derivatives of carbohydrates, proteins, nucleosides, and lipids are widely distributed in nature. These phosphorylated/polyphosphorylated derivatives play a dominant role in the physiology of cells by virtue of their unique interaction with target enzymes and receptors, and thus have a wide range of potential therapeutic applications.

For example, 2,3-Bisphospho-D-glycereate (BPG) is a natural allosteric effecter of hemoglobin in mammalian erythrocytes and regulates the affinity of oxygen. Phytic acid, also known as myo-inositol hexakisphosphate, is 1000 fold more potent than BPG, and can displace Hb-bound BPG. Further, many inositol-based phosphates have roles as second messengers. For instance the formation of D-myo-inositol 1,4,5-trisphosphate [I(1,4,5)P3] from membrane bound phosphatidylinositol bisphosphate is crucial for cellular signaling processes. I(1,4,5)P3 activates many calcium-dependent enzymes in cells by mobilizing the Ca⁺² ions from the intracellular storage.

Further still, phospholipid second messengers link upstream cellular receptors with downstream activities including proliferation, survival, chemotaxis, cellular trafficking, motility, metabolism, inflammatory and allergic responses, as well as transcription and translation. For example, phosphotidylinositol 3-kinases (PI3Ks) catalyze the transfer of phosphate to the D-3′ position of inositol lipids to produce phosphoinositol-3-phosphate (PIP), phosphoinositol-3,4-diphosphate (PIP2), and phosphoinositol-3,4,5-triphosphate (PIP3), which, in turn, act as second messengers in signaling cascades. The products of these cascades recruit proteins containing pleckstrin homology, FYVE, PHox and other phospholipid-binding domains, into a variety of signaling complexes often at the plasma membrane.

Thus, phosphorylated and pyrophosphate derivatives of polyols, and structural derivatives of these compounds, are potential therapeutic agents that display a broad spectrum of biological activities and provide a number of valuable pharmaceutical applications.

### SUMMARY OF THE INVENTION

The present invention provides phosphorylated and pyrophosphate derivatives of polyols, and structural derivatives of these compounds, and provides pharmaceutical compositions comprising the same. The compounds and compositions disclosed herein have various biological activities, including for example, as allosteric effectors of hemoglobin and/or as kinase inhibitors. The present invention further provides methods for therapy in human or mammalian patients, and methods for synthesis of biologically active compounds and their intermediates.

In one aspect, the invention provides a method for treating a patient having a condition associated with PI3 kinase activity. The invention comprises administering to the patient an effective amount of a phosphorylated and/or pyrophosphate derivative of a polyol, and other structural derivatives, described herein.

In this aspect, the patient's condition may be associated with, or characterized by, an amplification, somatic mutation, chromosomal rearrangement, overexpression, or overactivity of a PI3K, including a class I PI3K, class II PI3K, class III, and/or class IV PI3K. In some embodiments, the patient has a mutation or alteration in a PI3K pathway gene, leading to constitutive or overactivity of the PI3K pathway. In certain embodiments, the condition is associated with, or is further associated with, an inactivation of the tumor suppressor PTEN.

In various embodiments, the condition is an allergic disease, inflammation, heart disease (e.g., congestive heart failure), autoimmunity, diabetes mellitus, or cancer. For example, where the condition is cancer, the cancer may be ovarian, cervical, endometrial, colorectal, breast, pancreatic, gastric, glioblastoma, melanoma, liver, prostate, leukemia, lymphoma, head and neck, gastric, kidney, or lung. Particularly where the condition is a cancer or tumor, the patient may be tested prior to therapy for an alteration, hyperactivity, or overexpression of a PI3K, or mutation or inactivation of PTEN. For example, the patient may be tested for one or more of an amplification, somatic mutation, chromosomal rearrangement, overexpression, or overactivity of a PI3K, including a class I PI3K, class II PI3K, class III, and/or class IV PI3K, or a mutation or alteration in a PI3K pathway gene, or an alteration or inactivation of PTEN, such that the appropriate therapeutic regimen is selected for the patient.

In certain embodiments, the method comprises administering a polyphosphorylated inositol having at least one internal pyrophosphate ring. For example, the compound may be myo-inositol 1,6;2,3;4,5 tripyrophosphate, or a pharmaceutically acceptable prodrug or salt thereof, including a calcium salt, sodium salt, or mixed calcium and sodium salt.

In certain embodiments, the compound has at least one pyrophosphate ring or derivative thereof. For example, in these embodiments, the compound may have the following structure:

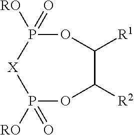

or a pharmaceutically acceptable salt, solvate, and/or prodrug thereof. R may be hydrogen, a cation, or any organic moiety. R¹ and R², taken together, may form a ring of 5 or 6 carbon atoms (which is optionally substituted), or alternatively, a heterocyclic 5-, 6-, or 7-membered ring (which is optionally substituted). At least one substituent of the ring may be a phosphate group or a pyrophosphate ring. Still further, R¹ and R², taken together, may form a polyol core, including: an aldose (e.g., Glyceraldehyde, Erythrose, Threose, Ribose, Arabinose, Xylose, Lyxose, Allose, Altrose, Glucose, Mannose, Gulose, Idose, Galactose, or Talose); an alditol (e.g., Glycerol, Erythritol, Threitol, Ribitol, Arabinitol, Xylitol, Allitol, Altritrol, Glucitol, Mannitol, Iditol, and Galactitol); or an aldaric acid (e.g., Glycaric acid, Tartaric acid, Ribaric acid, Arabinaric acid, Xylaric acid, Allaric acid, Altraric acid, Glucaric acid, Mannaric acid, and Galactaric acid). The aldose, aldotol, or aldaric acid may be further phosphorylated (e.g., having one, two, three, or four phosphate groups), and in certain embodiments, to include at least one additional pyrophosphate ring. Alternatively, at least one or both of R¹ and/or R² may be carboxy or an ester thereof of from 1 to 10 carbon atoms. Alternatively or in addition, one of R¹ and R² may form a pyrophosphate ring, which may be further substituted, e.g., by a carboxy group or ester thereof of from 1 to 10 carbon atoms.

In these or other embodiments, the patient is treated with one or more additional agents that act synergistically with PI3K inhibition. For example, in order to avoid drug resistance that may develop or exist as a result of pathway redundancy or molecular cross-talk, the invention may involve the use of the compounds described herein together in a treatment regimen or “cocktail” of kinase inhibitors, such as one or more PI3K pathway inhibitors, or one or more receptor tyrosine kinase inhibitors. In still other embodiments, the invention may involve the use of the compounds described herein together in a treatment regimen or “cocktail” of active agents that activate the apoptotic machinery, including conventional chemotherapeutic agents that act synergistically with PI3K inhibitors.

In certain embodiments, the compound, alternatively or in addition to inhibition of PI3K, also acts as an allosteric effector of hemoglobin, to enhance the delivery of oxygen to tissues. For example, where the condition is cancer, oxygenation of the tumor may result in increased sensitivity to radiation or increased chemosensitivity, or may reduce angiogenic and/or metastatic potential of the tumor. Where the condition is heart failure (e.g., congestive heart failure), the compound may increase the efficiency of oxygen delivery to body tissues, including the heart, to thereby ameliorate or slow progression of the disease.

In other aspects, the invention provides novel compounds for treating disease. In various embodiments, such compounds may act allosteric effectors of hemoglobin and/or as kinase (e.g., PI3K) inhibitors. Such compounds include phosphorylated and/or pyrophosphorylated derivatives of aldoses, including aldose-containing disacchararides and oligosaccharides. In still other embodiments, the compound is a phosphorylated or pyrophosphorylated derivative of an alditol, aldaric acid, or aldonic acid. The phosphorylated derivatives include polyphorphorylated derivatives, as well as pyrophosphate derivatives including one or more internal pyrophosphate rings.

### DESCRIPTION OF THE FIGURES

FIG. 1A-D illustrates exemplary polyols and related compounds (in Fischer representation when applicable). FIG. 1A illustrates exemplary aldoses (monosaccharides), for use in creating phosphorylated derivatives. FIG. 1B illustrates aldose-containing disaccharides for use in creating phosphorylated derivatives in accordance with the invention. FIG. 1C illustrates aldose-containing trisaccharides for use in creating phosphorylated derivatives in accordance with the invention. FIG. 1D illustrates a higher oligosaccharide for use in creating phosphorylated derivatives.

FIG. 2A-C illustrates various polyol derivatives. FIG. 2A illustrates exemplary alditols for creating phosphorylated derivatives in accordance with the invention. FIG. 2B illustrates exemplary aldaric acids for creating phosphorylated derivatives in accordance with the invention. FIG. 2C illustrates exemplary aldonic acids for creating phosphorylated derivatives in accordance with the invention.

### DETAILED DESCRIPTION OF THE INVENTION

In one aspect, the invention provides a method for treating a patient having a condition associated with PI3K activity or PI3K pathway activity, and use of a compound described herein for treatment of such condition or disease, including for the manufacture of a medicament for treatment of such condition or disease. In these embodiments, the patient's condition may be associated with, or characterized by, an amplification, somatic mutation, chromosomal rearrangement, overexpression, or overactivity of a PI3K, including a class I PI3K, class II PI3K, class III, and/or class IV PI3K, or alteration in a PI3K pathway gene. In various embodiments, the condition is an allergic disease, inflammation, heart disease (including congestive heart failure), autoimmunity, diabetes mellitus, or cancer. For example, where the condition is cancer, the cancer may be ovarian, endometrial, cervical, colorectal, gastric, breast, pancreatic, glioblastoma, melanoma, liver, prostate, leukemia, lymphoma, head and neck, gastric, kidney, or lung, including NSCLC. In certain embodiments, the condition is hepatoma, melanoma, pancreatic adenocarcinoma, and colon carcinoma. In certain embodiments, the condition is a lymphoma, such as non-hodgkin's lymphoma or chronic lymphocytic leukemia.

Of the two Class I PI3Ks, Class IA PI3Ks are heterodimers composed of a catalytic p110 subunit (α, β, δ isoforms) constitutively associated with a regulatory subunit that can be p85α, p55α, p50α, p85β, or p55γ. The Class 1B sub-class has one family member, a heterodimer composed of a catalytic p110γ subunit associated with one of two regulatory subunits, p101 or p84. The modular domains of p85/55/50 subunits include Src Homology (SH2) domains that bind phosphotyrosine residues in a specific context on an activated receptor and cytoplasmic tyrosine kinases resulting in activation and localization of Class 1A PI3Ks. Class 1B PI3K is activated directly by G protein-coupled receptors that bind a diverse repertoire of peptide and non-peptide ligands. PI3 kinases, including classes I-IV and associated genes, are described in WO 2005/091849, which description is hereby incorporated by reference.

Various alterations in PI3K are known to be associated with disease, including cancer. Exemplary mutations of the PIK3CA gene, which codes for the p110-α subunit of class 1A PI3K, are described in WO 2005/091849, which is hereby incorporated by reference in its entirety. For example, the tumor or cancer cells may have a mutation in the PIK3CA helical region (nt 1567-2124) or the kinase domain (nt 2095-3096). In certain embodiments, mutations may be in one or more of exons 1, 2, 4, 5, 7, 9, 13, 18, and 20 of the PI3KCA gene. Exemplary mutations include mutations at positions 1624 (e.g., G1624A), 1633 (e.g., G1633A), 1636 (e.g., C1636A), and 3140 (e.g., A3140G), 113 (e.g., G113A), 1258 (e.g., T1258C), 3129 (e.g., G3129T), 3139 (e.g., C3139T), and 2702 (e.g., G2702T) of the PIK3CA coding sequence. The condition may further be associated with alterations in any one or more of the class IA-encoding genes PIK3CB and PIK3CD; the class IIB-encoding gene PIK3CG; one or more of the class II-encoding genes PIK3C2A, PIK3C2B and/or PIK3C2G; the class III-encoding gene PIK3C3, and/or one or more of the class IV-encoding genes: ATM, ATR, FRAP1, SMG1, PRKDC, and/or TRRAP. Primers for detecting such alterations have also been described, and are disclosed in WO 2005/091849.

In these or other embodiments, the patient has one or more alterations in a PI3K pathway gene. Such mutations associated with cancer are described in WO 2006/127607, which is hereby incorporated by reference in its entirety. For example, the alteration in PI3K activity may be indicative of a somatic mutation or alteration in one or more of PDK1, AKT2, PAK-4, MARK3, MYLK2, CDC7, and PD1K1L.

In certain embodiments, the condition is associated with, or is further associated with, an inactivation of the tumor suppressor PTEN. Alterations or inactivations of the PTEN tumor suppressor gene are described for example in U.S. Pat. No. 7,129,040, which is hereby incorporated by reference.

Particularly where the condition is a cancer or tumor, the patient may be tested prior to therapy for an alteration, hyperactivity, or overexpression of a PI3K or PI3K pathway gene, or mutation or inactivation of PTEN. For example, the patient may be tested for one or more of an amplification, somatic mutation, chromosomal rearrangement, overexpression, or overactivity of PI3K or the PI3K pathway, including a class I PI3K, class II PI3K, class III, and/or class IV PI3K, or a mutation of PTEN tumor suppressor, such that the appropriate therapeutic regimen is selected. Such test may be conducted on the patient's cancer cells (e.g., a surgical specimen or biopsy), or cells cultured therefrom, and may be performed with any method known in the art, including nucleic acid sequencing, PCR, RT-PCR, or other detection platforms. Suitable detection platforms are further described in WO 2005/091849, WO 2006/127607, and U.S. Pat. No. 7,129,040, each of which is hereby incorporated by reference in its entirety. Briefly, such detection platforms include, without limitation, allele-specific or mutation-specific hybridization, allele-specific or mutation-specific amplification, primer extension, mutant-specific antibodies, RT-PCR (e.g., Taq-Man), and nucleic acid sequencing of allele-specific regions or regions known to harbor mutations associated with cancer. Where the presence of a mutation indicative of PI3K or PI3K activity is detected, the patient's treatment comprises an active agent disclosed herein for inhibiting PI3K pathway activity.

The invention comprises administering to the patient an effective amount of a polyphosphate and/or pyrophosphate derivative of a polyol, or structural mimetic described herein. Generally, the compound has at least one pyrophosphate ring, or structural derivative (structural mimic) thereof.

In certain embodiments, the compound is a polyphosphorylated inositol having at least one internal pyrophosphate ring. For example, the compound may be myo-inositol 1,6;2,3;4,5 tripyrophosphate, or a pharmaceutically acceptable prodrug or salt thereof, as described, for example, in U.S. Pat. No. 7,618,954, which is hereby incorporated by reference. Exemplary salts include a calcium salt, sodium salt (e.g., hexasodium salt), or mixed calcium and sodium salt (e.g., monocalcium/tetrasodium salt). Exemplary salts are disclosed in WO 2009/145751, which is hereby incorporated by reference.

In certain embodiments, the compound has at least one pyrophosphate ring or derivative thereof. For example, the compound may have the following structure:

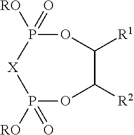

or a pharmaceutically acceptable salt, solvate, and/or prodrug thereof. In the above structure, R may be hydrogen, a cation, or any organic moiety; R¹ and R², taken together, may form a ring of 5 or 6 carbon atoms (which is optionally substituted), or alternatively, a heterocyclic 5-, 6-, or 7-membered ring (which is optionally substituted). At least one substituent of the ring may be a phosphate group or pyrophosphate ring (or “internal” pyrophosphate ring formed with a phosphate group at a neighboring position). Still further, R¹ and R², taken together, may form polyol core, including: an aldose (e.g., Glyceraldehyde, Erythrose, Threose, Ribose, Arabinose, Xylose, Lyxose, Allose, Altrose, Glucose, Mannose, Gulose, Idose, Galactose, or Talose); an alditol (e.g., Glycerol, Erythritol, Threitol, Ribitol, Arabinitol, Xylitol, Allitol, Altritrol, Glucitol, Mannitol, Iditol, and Galactitol); or an aldaric acid (e.g., Glycaric acid, Tartaric acid, Ribaric acid, Arabinaric acid, Xylaric acid, Allaric acid, Altraric acid, Glucaric acid, Mannaric acid, and Galactaric acid). The aldose, aldotol, or aldaric acid may be further phosphorylated (e.g., having one, two, three, or four phosphate groups), and in certain embodiments, includes at least one additional pyrophosphate ring. Alternatively, at least one or both of R¹ and/or R² may be carboxy or an ester thereof of from 1 to 10 (e.g., from 1 to 4) carbon atoms. Alternatively or in addition, one of R¹ and R² may form a pyrophosphate ring, which may be further substituted, e.g., by a carboxy group or ester thereof of from 1 to 10 (e.g., from 1 to 4) carbon atoms.

Alternatively, the method may include administering any compound described herein, including a compound of formulas II, IIa, IIb, IIc, IId, or III, or a compound of Tables 1 or 2.

The compound may be used in a treatment regimen, or chemotherapeutic cocktail. In these or other embodiments, the patient is treated with one or more additional agents that act additively or synergistically with PI3K inhibition. See, for example, Premkumar et al., Synergistic augmentation of vincristine-induced cytotoxicity by phosphatidylinositol 3-kinase inhibitor in human malignant glioma cells: evidence for the involvement of p38 and ERK signaling pathways, Cancer Therapy 3:407-418 (2005); Olcay et al., Drug delivery and drug resistance: EGFR-tyrosine kinase inhibitors in non-small cell lung cancer, The Open Lung Cancer Journal 3:26-33 (2010. For example, in order to avoid drug resistance that may develop or exist as a result of pathway redundancy or molecular cross-talk, the invention may involve the use of the compounds described herein together in a treatment regimen or “cocktail” of kinase inhibitors, such as one or more PI3K pathway inhibitors or one or more receptor tyrosine kinase inhibitors. In various embodiments the compounds described herein are combined with agents that target one or more of EGFR, JAK2, mTOR, CK2, MEK, and HER2. In still other embodiments, the invention may involve the use of the compounds described herein together in a treatment regimen or “cocktail” of inhibitors that activate or induce the apoptotic machinery, including the molecular targets BCL-xL and HDAC. Such therapeutic agents that may act synergistically or additively with the compounds described herein include one or more of docetaxel, paclitaxel, doxorubicin, epirubicin, rapamycin, vincristine, erlotinib, gefitinib, lapatinib, cetuximab, panitumumab, imatinib, apigenin, 1-alpha, 25-dihydroxyvitamin D3, trastuzumab, ABT-737, and gemcitabine.

In certain embodiments, the compound, alternatively or in addition to inhibition of PI3K, acts as an allosteric effector of hemoglobin, to enhance the delivery of oxygen to tissues. For example, where the condition is cancer, oxygenation of the tumor may result in increased sensitivity to radiation or increased chemosensitivity, or may reduce angiogenic and/or metastatic potential of the tumor. Without being bound by theory, in these embodiments, it is believed that the active agent works in part by suppression of HIF1α, a transcription factors that responds to change in available oxygen in the cellular environment. These embodiments for allosteric effectors of hemoglobin are described in one or more of U.S. Pat. No. 7,745,423, U.S. Pat. No. 7,618,954, and U.S. 2008/0200437, each of which are hereby incorporated by reference in their entirety. Where the condition is heart failure, such as congestive heart failure, the compound may increase the efficiency of oxygen delivery to body tissues, including the heart, to thereby ameliorate or slow progression of the disease, as described in U.S. Pat. No. 7,618,954, which are hereby incorporated by reference in their entirety. Additional conditions, for which the allosteric effectors of hemoglobin find use, include anemia, hypoxia, and Alzheimers Disease.

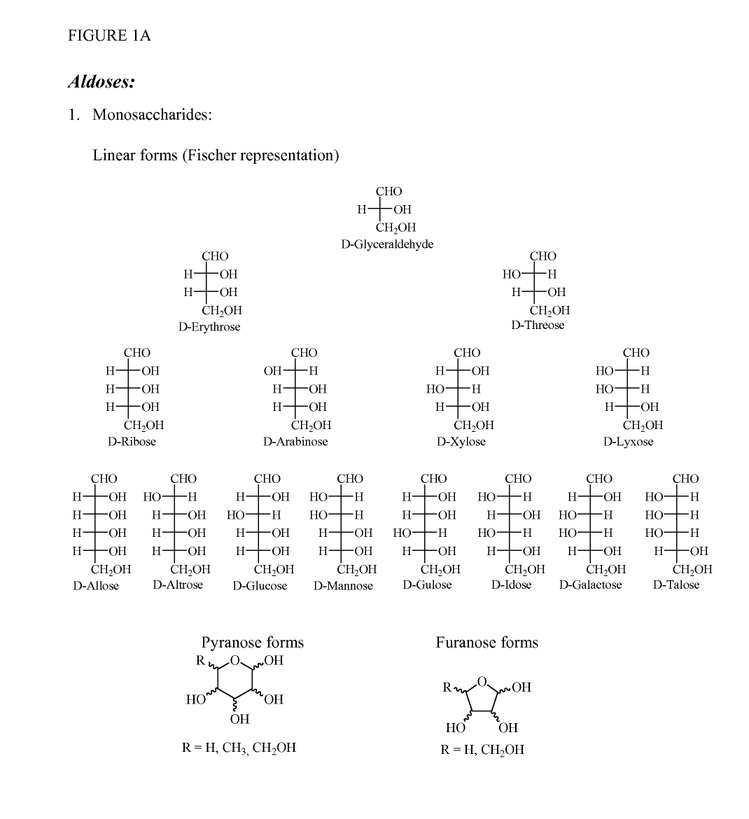

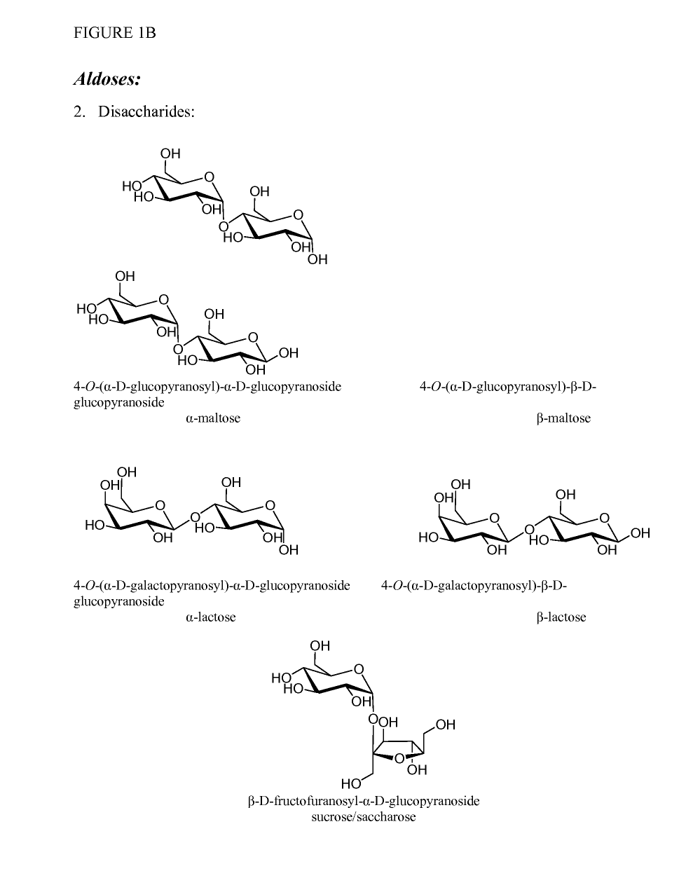

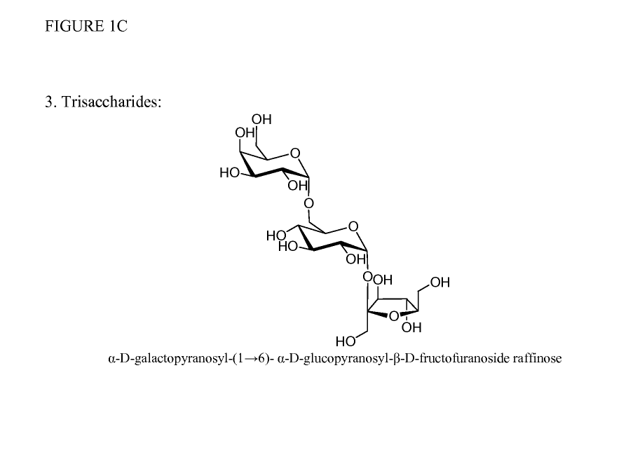

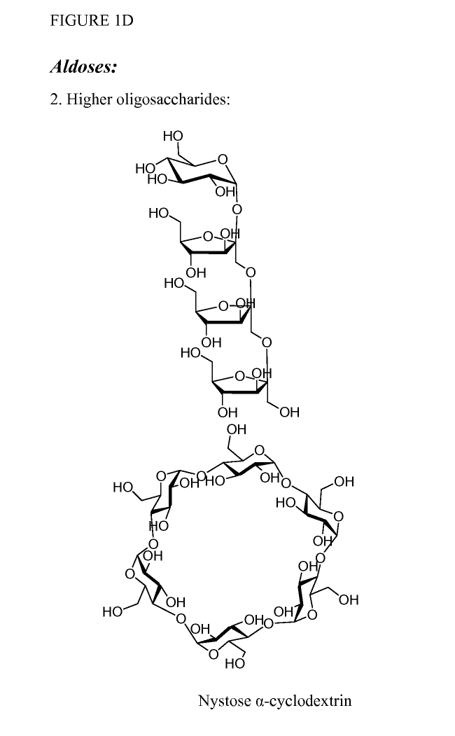

In other aspects, the invention provides novel compounds for treating disease, including as allosteric effectors of hemoglobin or as kinase inhibitors. Such compounds include phosphorylated derivatives of aldoses, including aldose-containing disacchararides and oligosaccharides. Exemplary aldoses, including aldose-containing disaccharide or trisaccharide are disclosed in FIG. 1. For example, the active agent may be a derivatized D-Glyceraldehyde, D-Erythrose, D-Threose, D-Ribose, D-Arabinose, D-Xylose, D-Lyxose, D-Allose, D-Altrose, D-Glucose, D-Mannose, D-Gulose, D-Idose, D-Galactose, or D-Talose. Such saccharide units are derivatized to be polyphosphorylated (e.g., having one, two, three, or four phosphate groups), and in certain embodiments, to include one or two pyrophosphate ring(s), including internal pyrophosphate rings.

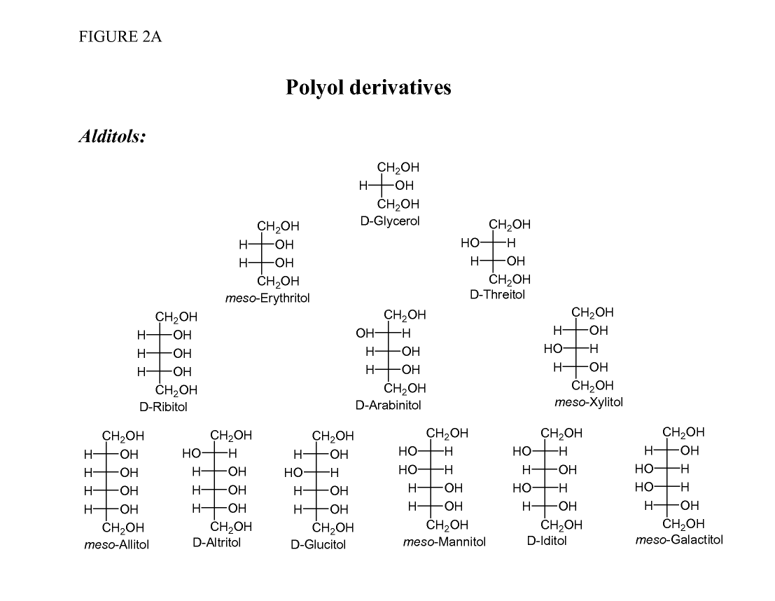

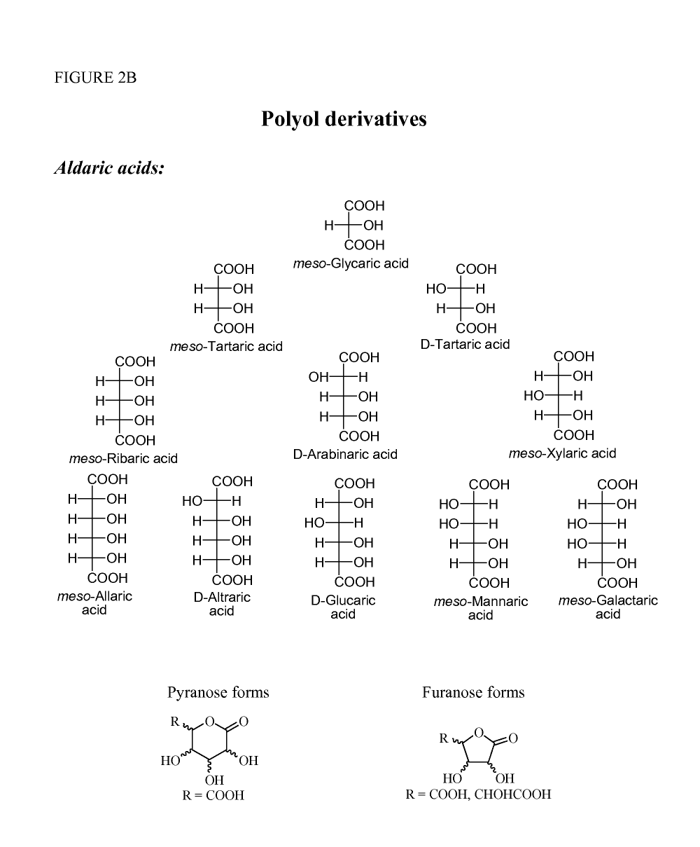

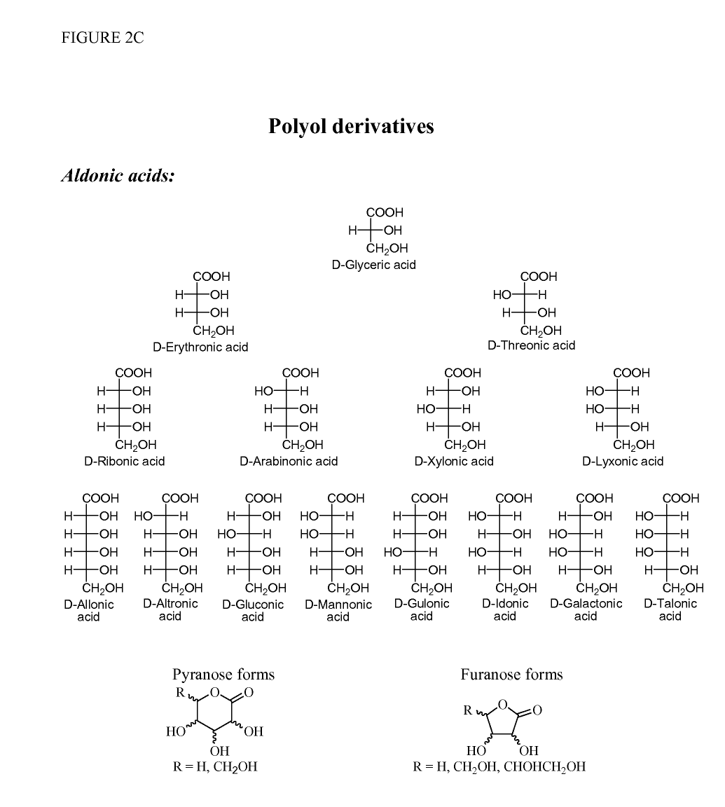

In some embodiments, the compounds include phosphorylated derivatives of an alditol, including alditol-containing disacchararides and oligosaccharides. Exemplary alditols are disclosed in FIG. 2. For example, the active agent may be a derivatized D-Glycerol, D-Erythritol, D-Threitol, D-Ribitol, D-Arabinitol, meso-Xylitol, meso-Allitol, D-Altritrol, D-Glucitol, meso-Mannitol, D-Iditol, and meso-Galactitol. Such saccharide units are derivatized to be polyphosphorylated (e.g., having one, two, three, or four phosphate groups), and in certain embodiments, to include one or two pyrophosphate ring(s), including internal pyrophosphate rings.

In some embodiments, the compounds include phosphorylated derivatives of an aldaric acid, including aldaric acid-containing disacchararides and oligosaccharides. Exemplary aldaric acids are disclosed in FIG. 2. For example, the active agent may be a derivatized meso-Glycaric acid, meso-Tartaric acid, D-Tartaric acid, meso-Ribaric acid, D-Arabinaric acid, meso-Xylaric acid, meso-Allaric acid, D-Altraric acid, D-Glucaric acid, meso-Mannaric acid, and meso-Galactaric acid. Such saccharide units are derivatized to be polyphosphorylated (e.g., having one, two, or three, or four phosphate groups), and in certain embodiments, to include at least one or two pyrophosphate ring(s) or internal pyrophosphate ring(s).

In various embodiments, the compound of the invention has the following structure:

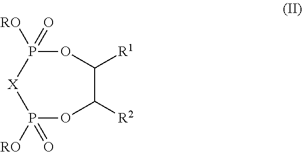

or a pharmaceutically acceptable salt, solvate, and/or prodrug thereof;

wherein,

X is O, S, NH, or NR^{a};

R¹ and R² are independently hydrogen, halo, R^{a}, —OR^{b}, —SR^{b}, —NR^{c}R^{c}, —CN, —OCN, —SCN, —NO, —NO₂, —N₃, —S(O)₂R^{b}, —S(O)₂NR^{b}, —S(O)₂O⁻, —S(O)₂OR^{b}, —OS(O)₂R^{b}, —OS(O)₂O⁻, −OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b}, or —NR^{b}C(NR^{b})NR^{c}R^{c}; or alternatively, R¹ and R², taken together with the atoms to which they are bonded, form a 4-, 5-, 6-, 7-, or 8-membered carbocyclyl or heterocyclyl which is optionally further substituted;

each R is independently hydrogen, a cation, or R^{a};

R^{a} is optionally substituted alkyl, optionally substituted alkenyl, optionally substituted alkynyl, optionally substituted carbocyclyl, optionally substituted heteroalkyl, optionally substituted heterocyclyl, optionally substituted aryl, optionally substituted arylalkyl, optionally substituted heteroaryl, and optionally substituted heteroarylalkyl;

each R^{b} is independently hydrogen, a cation, or R^{a};

each R^{c} is independently hydrogen, a cation, or R^{a}; or alternatively, two R^{c}, taken together with the nitrogen atom to which they are bonded, form a 4-, 5-, 6- or 7-membered heterocyclyl which is optionally further substituted; and

with the proviso that R¹ and R² are not both hydrogen.

In one embodiment of the present invention, Formula (II) does not include a compound wherein R¹ and R², taken together with the atoms to which they are bonded, form a inositol backbone structure.

In one embodiment of Formula (II), at least one of R¹ and R² is —S(O)₂R^{b}, —S(O)₂NR^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, NR^{b}C(O)R^{c}R^{c}, —NR^{b}C(NR^{b})R^{b}, or —NR^{b}C(NR^{b})NR^{c}R^{c}; and the other R¹ or R², Ra, Rb, and Rc are the same as defined above.

In one embodiment of Formula (II), R¹ and R² are independently —S(O)₂R^{b}, —S(O)₂NR^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)R^{c}R^{c}, —NR^{b}C(NR^{b})R^{b}, or —NR^{b}C(NR^{b})NR^{c}R^{c}; and R^{a}, R^{b}, and R^{c} are the same as defined above.

In one embodiment of Formula (II), one of R¹ and R² is —S(O)₂R^{b}, —S(O)₂NR^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)R^{c}R^{c}, —NR^{b}C(NR^{b})R^{b}, or —NR^{b}C(NR^{b})NR^{c}R^{c}; the other R¹ or R² is an alkyl substituted by a pyrophosphate group and a group selected from —S(O)₂R^{b}, —S(O)₂NR^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)R^{c}R^{c}, —NR^{b}C(NR^{b})R^{b}, and —NR^{b}C(NR^{b})NR^{c}R^{c}; and the other R¹ or R², R^{a}, R^{b}, and R^{c} are the same as defined above.

In one embodiment of Formula (II), R¹ and R², taken together with the atoms to which they are bonded, form a 7-membered heterocyclyl. In one embodiment, the 7-membered heterocyclyl is a pyrophosphate ring.

In one embodiment of Formula (II), R¹ and R², taken together with the atoms to which they are bonded, form a 5-, 6-, 7-, or 8-membered carbocyclyl which is optionally further substituted. In one embodiment, R¹ and R², taken together with the atoms to which they are bonded, form a 6-membered carbocyclyl which is further substituted a pyrophosphate group.

In certain embodiments, the compound may have the following structural Formula (III):

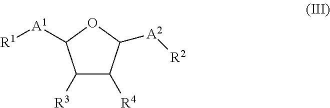

or a pharmaceutically acceptable salt, solvate, and/or prodrug thereof;
wherein,

A¹ and A² are independently C1 to C3 optionally substituted alkyl, optionally substituted alkenyl, optionally substituted alkynylene;

R¹, R², R³, and R⁴ are independently —P(O)(O⁻)₂, —P(O)(OR)(O⁻), or —P(O)(OR)(OR); or alternatively, R1 and R3, R3 and R4, or R2 and R4, taken together with the atoms to which they are attached, form a pyrophosphate group; and

each R is independently hydrogen, cation, optionally substituted alkyl, optionally substituted alkenyl, optionally substituted alkynyl, optionally substituted carbocyclyl, optionally substituted heteroalkyl, optionally substituted heterocyclyl, optionally substituted aryl, optionally substituted arylalkyl, optionally substituted heteroaryl, or optionally substituted heteroarylalkyl.

In one embodiment of Formula (III), A¹ and A² are —CH₂—.

In other aspects, the invention provides novel compounds for treating disease. According to this aspect, the compounds may have a structure selected from:

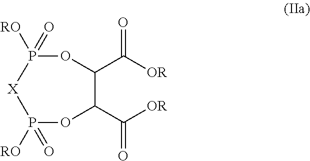

or a pharmaceutically acceptable salt, solvate, and/or prodrug thereof;
wherein

X is O, S, NH, or NR; and

each R is independently hydrogen, a cation, optionally substituted alkyl, optionally substituted alkenyl, optionally substituted alkynyl, optionally substituted carbocyclyl, optionally substituted heteroalkyl, optionally substituted heterocyclyl, optionally substituted aryl, optionally substituted arylalkyl, optionally substituted heteroaryl, or optionally substituted heteroarylalkyl.

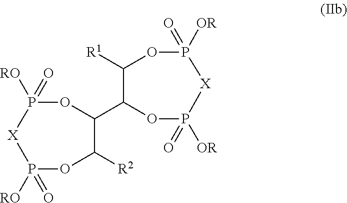

or a pharmaceutically acceptable salt, solvate, and/or prodrug thereof;
wherein

each X is independently O, S, NH, or NR;

R¹ and R² are independently —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b}, or —NR^{b}C(NR^{b})NR^{c}R^{c}; and R^{a}, R^{b}, and R^{c} are the same as defined above in Formula (II); and

each R is independently hydrogen, a cation, optionally substituted alkyl, optionally substituted alkenyl, optionally substituted alkynyl, optionally substituted carbocyclyl, optionally substituted heteroalkyl, optionally substituted heterocyclyl, optionally substituted aryl, optionally substituted arylalkyl, optionally substituted heteroaryl, or optionally substituted heteroarylalkyl.

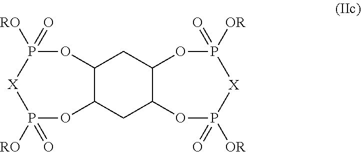

or a pharmaceutically acceptable salt, solvate, and/or prodrug thereof;
wherein

X is O, S, NH, or NR; and

each R is independently hydrogen, a cation, optionally substituted alkyl, optionally substituted alkenyl, optionally substituted alkynyl, optionally substituted carbocyclyl, optionally substituted heteroalkyl, optionally substituted heterocyclyl, optionally substituted aryl, optionally substituted arylalkyl, optionally substituted heteroaryl, or optionally substituted heteroarylalkyl.

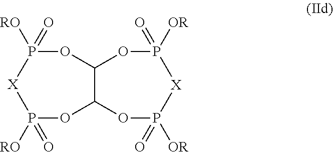

or a pharmaceutically acceptable salt, solvate, and/or prodrug thereof;
wherein

X is O, S, NH, or NR; and

each R is independently hydrogen, a cation, optionally substituted alkyl, optionally substituted alkenyl, optionally substituted alkynyl, optionally substituted carbocyclyl, optionally substituted heteroalkyl, optionally substituted heterocyclyl, optionally substituted aryl, optionally substituted arylalkyl, optionally substituted heteroaryl, or optionally substituted heteroarylalkyl.

The descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The compounds of the invention often have ionizable groups so as to be capable of preparation as salts. In that case, wherever reference is made to the compound, it is understood in the art that a pharmaceutically acceptable salt may also be used. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art. In some cases, the compounds may contain both an acidic and a basic functional group, in which case they may have two ionized groups and yet have no net charge. Standard methods for the preparation of pharmaceutically acceptable salts and their formulations are well known in the art, and are disclosed in various references, including for example, “Remington: The Science and Practice of Pharmacy”, A. Gennaro, ed., 20th edition, Lippincott, Williams & Wilkins, Philadelphia, Pa.

“Solvate”, as used herein, means a compound formed by solvation (the combination of solvent molecules with molecules or ions of the solute), or an aggregate that consists of a solute ion or molecule, i.e., a compound of the invention, with one or more solvent molecules. When water is the solvent, the corresponding solvate is “hydrate”. Examples of hydrate include, but are not limited to, hemihydrate, monohydrate, dihydrate, trihydrate, hexahydrate, etc. It should be understood by one of ordinary skill in the art that the pharmaceutically acceptable salt, and/or prodrug of the present compound may also exist in a solvate form. The solvate is typically formed via hydration which is either part of the preparation of the present compound or through natural absorption of moisture by the anhydrous compound of the present invention.

The term “ester” means any ester of a present compound in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The hydrolysable esters of the present compounds are the compounds whose carboxyls are present in the form of hydrolysable ester groups. That is, these esters are pharmaceutically acceptable and can be hydrolyzed to the corresponding carboxyl acid in vivo. These esters may be conventional ones, including lower alkanoyloxyalkyl esters, e.g. pivaloyloxymethyl and 1-pivaloyloxyethyl esters; lower alkoxycarbonylalkyl esters, e.g., methoxycarbonyloxymethyl, 1-ethoxycarbonyloxyethyl, and 1-isopropylcarbonyloxyethyl esters; lower alkoxymethyl esters, e.g., methoxymethyl esters, lactonyl esters, benzofuran keto esters, thiobenzofuran keto esters; lower alkanoylaminomethyl esters, e.g., acetylaminomethyl esters. Other esters can also be used, such as benzyl esters and cyano methyl esters. Other examples of these esters include: (2,2-dimethyl-1-oxypropyloxy)methyl esters; (1RS)-1-acetoxyethyl esters, 2-[(2-methylpropyloxy)carbonyl]-2-pentenyl esters, 1-[[(1-methylethoxy)carbonyl]-oxy]ethyl esters; isopropyloxycarbonyloxyethyl esters, (5-methyl-2-oxo-1,3-dioxole-4-yl) methyl esters, 1-[[(cyclohexyloxy)carbonyl]oxy]ethyl esters; 3,3-dimethyl-2-oxobutyl esters.

The term “prodrug” refers to a precursor of a pharmaceutically active compound wherein the precursor itself may or may not be pharmaceutically active but, upon administration, will be converted, either metabolically or otherwise, into the pharmaceutically active compound or drug of interest. For example, prodrug can be an ester, ether, or amide form of a pharmaceutically active compound. Various types of prodrug have been prepared and disclosed for a variety of pharmaceuticals. See, for example, Bundgaard, H. and Moss, J., J. Pharm. Sci. 78: 122-126 (1989).

As used herein, “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

“Excipient” refers to a diluent, adjuvant, vehicle, or carrier with which a compound is administered.

The compounds and compositions described herein can be provided as physiologically acceptable formulations, and can be administered by standard routes. For example, the combinations may be administered, for example, by the oral, rectal, or parenteral (e.g., intravenous, subcutaneous or intramuscular) route. In addition, the combinations may be incorporated into polymers allowing for sustained release, the polymers being implanted in the vicinity of where delivery is desired, for example, at the site of a tumor, or into a cavity or blood vessel that will lead to easy delivery to the place to be treated. The dosage of the composition will depend on the condition being treated, the particular derivative used, and other clinical factors such as weight and condition of the patient and the route of administration of the compound.

The formulations in accordance with the present invention can be administered in the form of tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

The formulations include those suitable for oral, rectal, nasal, inhalation, topical (including dermal, transdermal, buccal and sublingual), vaginal, parenteral (including subcutaneous, intramuscular, intravenous, intraperitoneal, intradermal, intraocular, intratracheal, and epidural) or inhalation administration. The formulations may conveniently be presented in unit dosage form and may be prepared by conventional pharmaceutical techniques.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface-active or dispersing agent. Molded tablets may be made by molding, in a suitable machine, a mixture of the powdered compound moistened with an inert liquid diluent. The tablets may optionally be coated or scored and may be formulated so as to provide a slow or controlled release of the active ingredient therein.

Formulations suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutically acceptable carrier. A preferred topical delivery system is a transdermal patch containing the ingredient to be administered.

Formulations for rectal administration may be presented as a suppository with a suitable base comprising, for example, cocoa butter and/or a salicylate.

Formulations suitable for nasal administration, wherein the carrier is a solid, include a coarse powder having a particle size, for example, in the range of 20 to 500 microns which is administered in the manner in which snuff is taken; i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing, in addition to the active ingredient, ingredients such as carriers as are known in the art to be appropriate.

Formulation suitable for inhalation may be presented as mists, dusts, powders or spray formulations containing, in addition to the active ingredient, ingredients such as carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in freeze-dried (lyophilized) conditions requiring only the addition of a sterile liquid carrier, for example, water for injections, immediately prior to use. Extemporaneous injection solutions and suspensions may be prepared from sterile powders, granules and tablets of the kinds previously described.

Preferred unit dosage formulations are those containing a daily dose or unit, daily sub-dose, as herein above recited, or an appropriate fraction thereof, of the administered ingredient.

### EXAMPLES

### Examples 1

### Synthesis

Phosphorylation of polyols and related compounds, by means of phosphoric acid based reagents yields (poly) phosphorylated derivatives. The latter are best isolated, purified and conserved as their sodium salts. Other salts, such as other metal cation or ammonium salts, may be prepared and serve similar or related purposes.

Such transformations apply to the polyol-derived polyhydroxylated molecules illustrated in FIGS. 1 and 2. The structures of a selection of polyols and related compounds of the invention are given in FIGS. 1 and 2. The disposition of hydroxyl groups —OH (or derived phosphorylated groups —OPO²⁻₃) is given following the Fischer representation.

The polyphosphates above may be converted into derivatives containing cyclic pyrophosphate groups by dehydration, using agents such as dicyclohexylcarbodiimide or related reagents.

This conversion may either be total or yield compounds containing both phosphate P and pyrophosphate PP functional groups. The compounds obtained are best isolated, purified and kept as their sodium salts. Other salts may be prepared and serve similar purposes. The fully phosphorylated compounds may lead to one or two pyrophosphate derivatives.

The pyrophosphates of the polyols and related compounds, such as those shown in FIGS. 1-2 may be converted into the corresponding phosphorimides or thiopyrophosphates by a sequence of opening/closing reactions. The Structure A shown below gives a representation of the phosphorimide (X═NR) and thiopyrophosphate (X═S) groups without indication of stereochemistry. For the phosphorimides, (Structure A, X═NR), the R group on the nitrogen N can be R═H or an organic residue, in particular a hydrocarbon chain CₙH₂ₙ₊₁, or a chain or a group containing heteroatoms, such as oxygen.

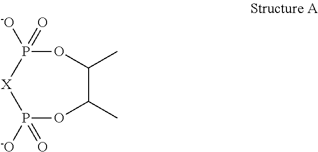

X═O pyrophosphate
X═NR phosphorimide
X═S thiopyrophosphate

The synthetic plan involved the synthesis of perphosphorylated polyols which also have the ability to form pyrophosphates, in particular cyclic pyrophosphates. A variety of commercially available or synthetic polyols were subjected to phosphorylation reaction in order to achieve the desired bis and tetrakis phosphorylated compounds.

The schemes for the synthesis and the biological experiments (in pure hemoglobin and whole blood) of these compounds are provided below.

### 1. Diethyl-L-tartrate

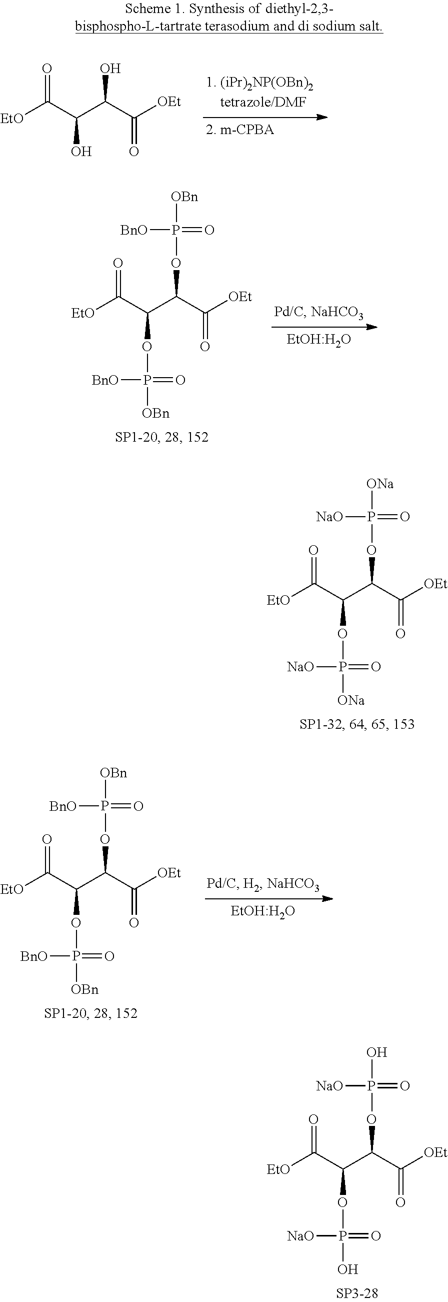

### 2. Dibutyl-L-tartrate

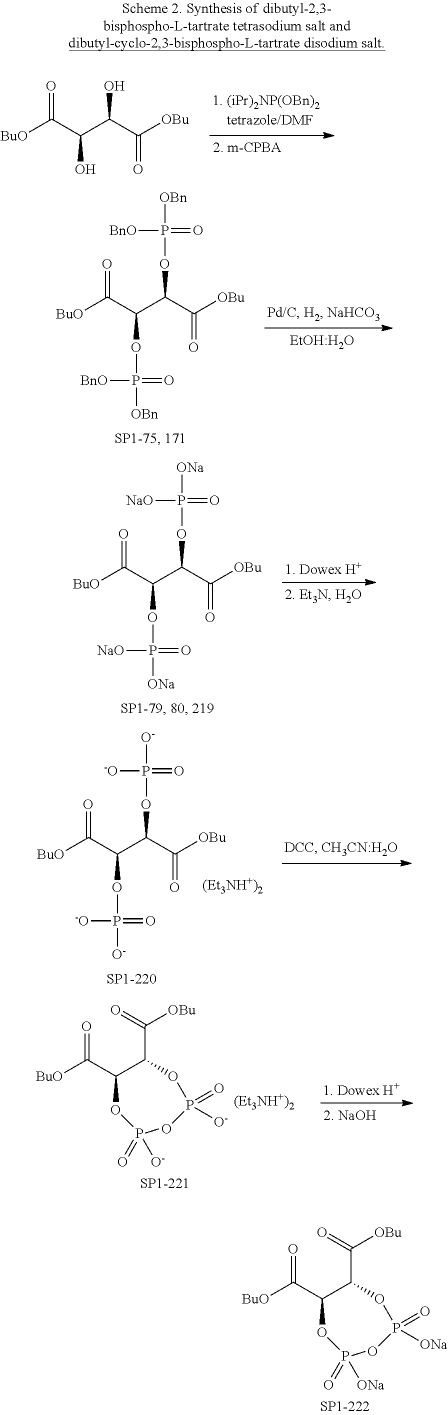

### 3. Dibenzyl-L-tartrate

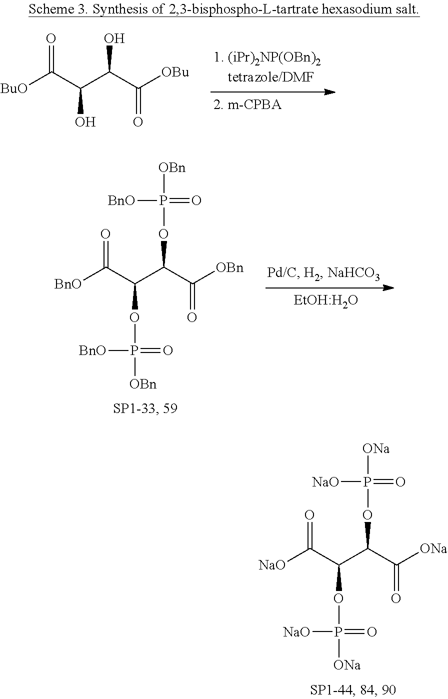

### 4. Dimethyl-meso-galactarate

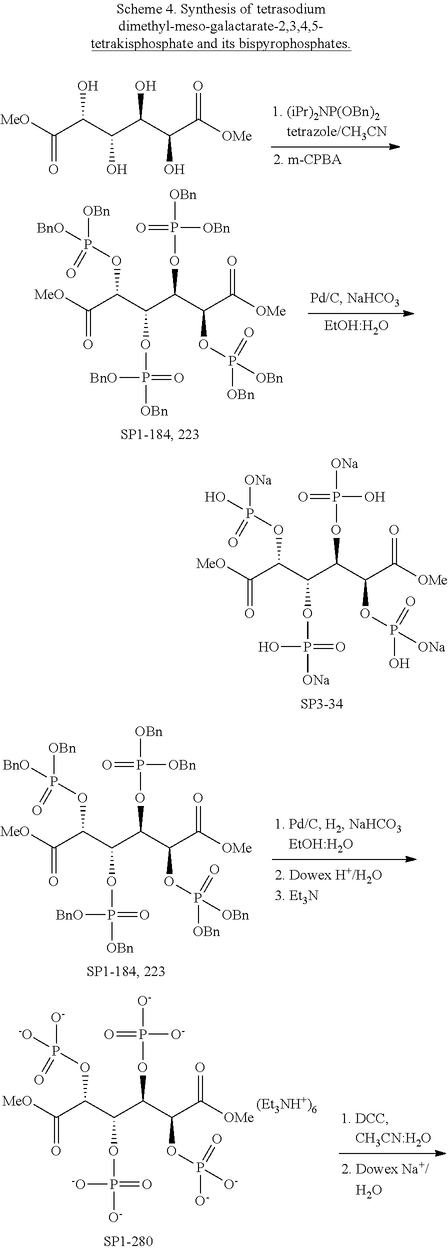

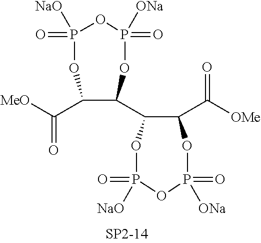

### 5. meso-Erythritol

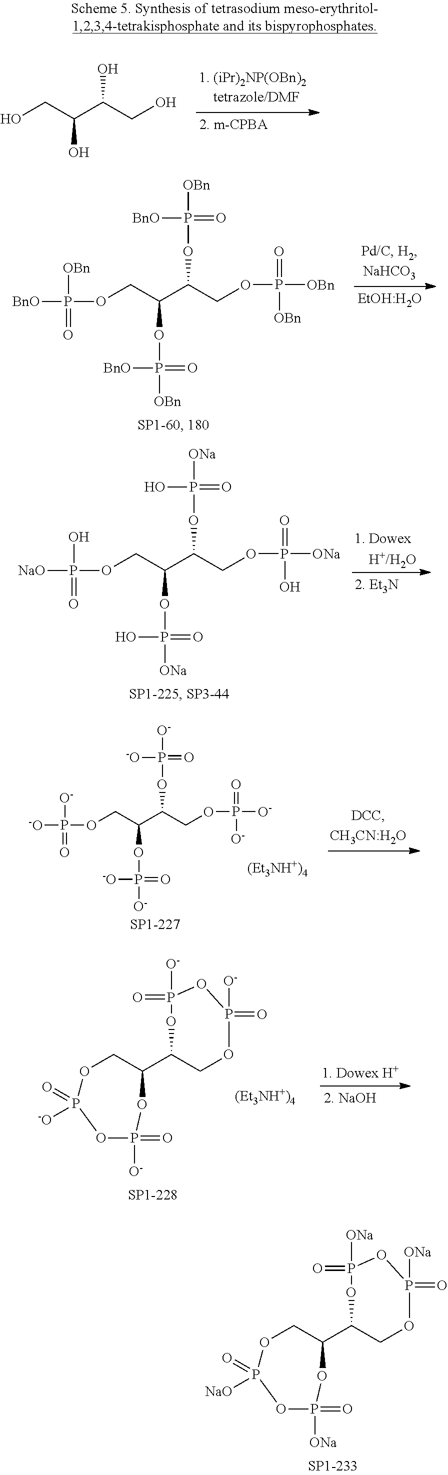

### 6. Pentaerythritol

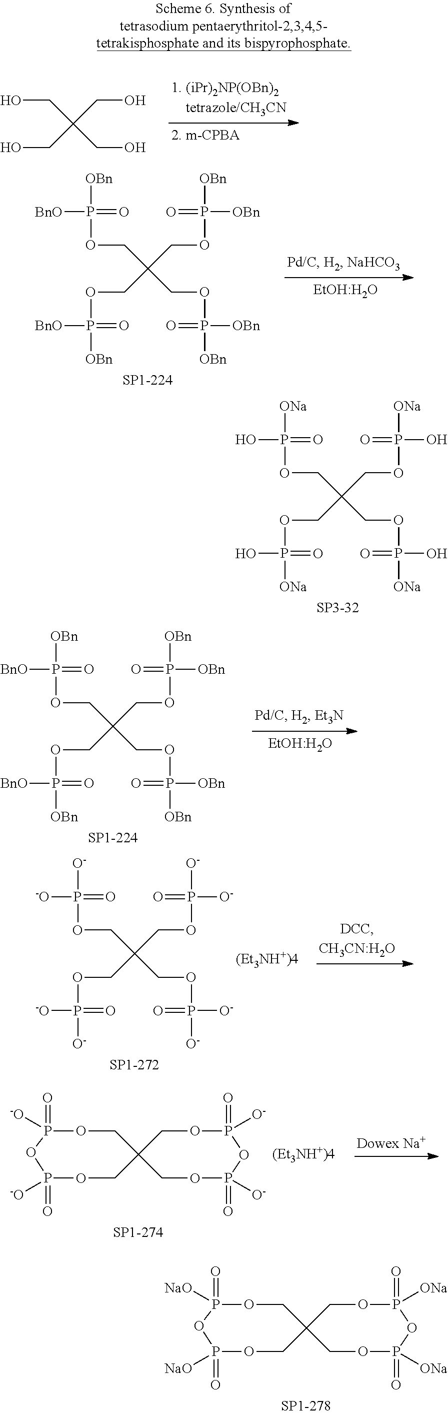

### 7. 2,5-Anhydro-D-mannitol

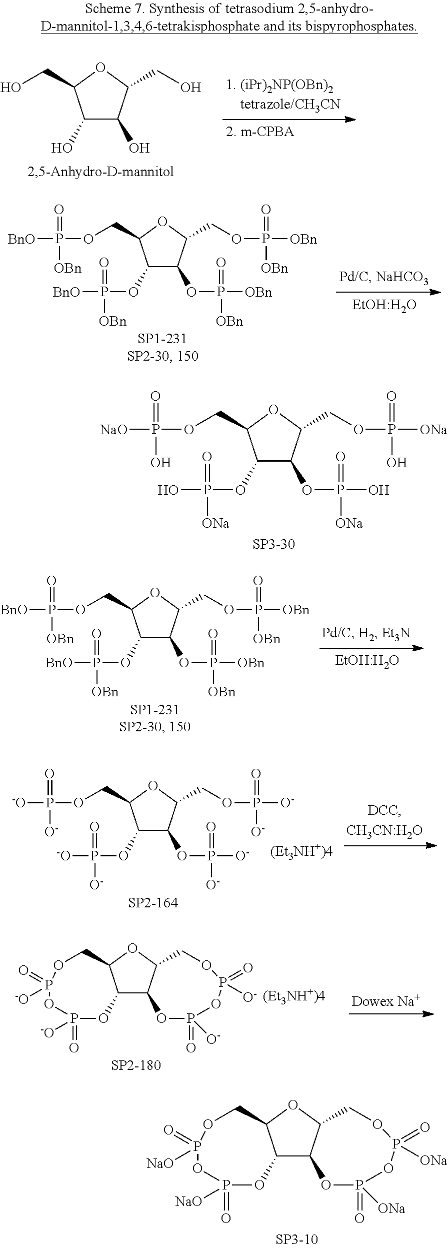

### Diethyl-2,3-bis(dibenzylphospho)-L-tartrate

To diethyl-L-tartrate (0.5 mL, 2.91 mmol, 1 eq), tetrazole (0.45 M in acetonitrile) (25.9 mL, 8.75 mmol, 3 eq) at room temperature was added followed by dibenzyl N,N-diisopropylphosphoramidite (2.94 mL, 8.75 mmol, 3 eq). After being stirred for 24 h at room temperature, the reaction mixture was cooled to −40° C., then 3-chloroperbenzoic acid (1.61 g, 9.34 mmol, 3.2 eq) was added portion wise and the reaction was allowed to stir from −40° C. to room temperature over a period of 12 h. Then the reaction mixture was diluted with EtOAc, washed with 10% Na₂SO₃, saturated NaHCO₃, brine then dried over Na₂SO₄ and concentrated in vacuo. The crude compound was purified by silica gel column chromatography (EtOAc/n-heptane, 10:90 to 30:70) to obtain diethyl-2,3-bis(dibenzylphospho)-L-tartrate as an oil (1.54 g, 72%).

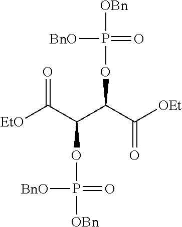 [SMILES: CCOC(=O)[C@H](OP(=O)(OCc1ccccc1)OCc1ccccc1)[C@@H](OP(=O)(OCc1ccccc1)OCc1ccccc1)C(=O)OCC]

TLC (SiO₂): R_{f}=0.22 (EtOAc/n-heptane, 70:30); [α]²⁰_{D} 5.0 (c 1.0, CHCl₃); IR: ν=3853, 3744, 3675, 3045, 2985, 1764, 1456, 1265, 1214, 1017, 957, 734, 699 cm⁻¹; ¹H NMR (CDCl₃, 400 MHz, 25° C.): δ=7.35-7.31 (m, 20H), 5.47 (dd, ³J_{HP}=8.5 Hz, ⁴J_{HP}=2.4 Hz, 2H), 5.25 (d of AB, J=11.6 Hz, ³J_{HP}=7.3 Hz, 2H), 5.19 (d of AB, J=11.6 Hz, ³J_{HP}=6.7 Hz, 2H), 5.01 (d, J=6.7 Hz, 4H), 4.18 (dq, J=10.7, 6.7 Hz, 2H), 4.04 (dq, J=10.7, 7.3 Hz, 2H), 1.18 (t, J=7.3 Hz, 6H); ¹³C NMR (CDCl₃, 100 MHz, 25° C.): δ=166.1 (d, ³J_{CP}=2.3 Hz, 2C), 135.6 (d, ³J_{CP}=7.6 Hz, 2C), 135.3 (d, ³J_{CP}=7.6 Hz, 2C), 128.4, 128.4, 128.4, 127.9, 127.83, 127.80, 127.76, 75.3 (dd, ²J_{CP}=7.2 Hz, ³J_{CP}=5.0 Hz, 2C), 69.8 (d, ²J_{CP}=5.3 Hz, 2C), 69.6 (d, ²J_{CP}=5.3 Hz, 2C), 62.6, 13.8; ³¹P NMR (CDCl₃, 162 MHz, 25° C.): δ=−2.08; HRMS (ESI): m/z: calcd for C₃₆H₄₀NaO₁₂P₂: 749.1887 [M+Na]⁺. found: 749.1919.

### Diethyl-2,3-bisphospho-L-tartrate tetrasodium salt

To a solution of diethyl-2,3-bis(dibenzylphospho)-L-tartrate (442 mg, 0.60 mmol, 1 eq) in EtOH:H₂O (1:1, 40 mL) was added 10% Pd/C (176 mg) and sodium bicarbonate (204 mg, 2.43 mmol, 4 eq). The solution was evacuated for few minutes, and then the reaction mixture was stirred at room temperature under an atmosphere of hydrogen for 12 h. The catalyst was filtered through a LCR/PTFE hydrophilic membrane, washed with 2×20 mL of EtOH:H₂O (1:1). The solvent was evaporated under reduced pressure and lyophilisation afforded diethyl-2,3-bisphospho-L-tartrate tetrasodium salt as a white solid (323 mg, 93%).

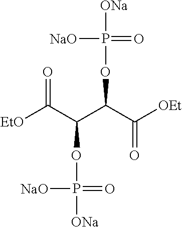

¹H NMR (D₂O, 400 MHz, 25° C.): δ=4.79 (dd, J=9.7, 2.2 Hz, 2H), 4.32 (dq, J=10.8, 7.5 Hz, 2H), 4.20 (dq, J=10.8, 7.0 Hz, 2H), 1.32 (t, J=7.5 Hz, 6H); ¹³C NMR (D₂O, 100 MHz, 25° C.): δ=171.7, 73.8 (dd, ²J_{CP}=6.1 Hz, ³J_{CP}=4.6 Hz, 2C), 62.7, 13.3; ³¹P NMR (D₂O, 162 MHz, 25° C.): δ=3.54; HRMS (ESI): m/z: calcd for C₈H₁₃Na₄O₁₂P₂: 454.9468 [M+H]⁺. found: 454.9409.

### Diethyl-2,3-bisphospho-L-tartrate disodium salt

To a solution of diethyl-2,3-bis(dibenzylphospho)-L-tartrate (1.5 g, 2.0 mmol, 1 eq) in EtOH:H₂O (1:1, 50 mL) was added 10% Pd/C (600 mg) and sodium bicarbonate (347 mg, 4.1 mmol, 2 eq). The solution was evacuated for few minutes, and then the reaction mixture was stirred at room temperature under an atmosphere of hydrogen for 9 h. The catalyst was filtered through a LCR/PTFE hydrophilic membrane, washed with 2×20 mL of EtOH:H₂O (1:1). The solvent was evaporated under reduced pressure and lyophilisation afforded diethyl-2,3-bisphospho-L-tartrate disodium salt as a white solid (824 mg, 97%).

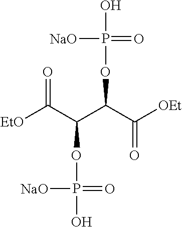

[α]²⁰_{D} 1.1 (c 1.0, H₂O); IR: ν=2984, 2769, 1760, 1275, 1209, 1082, 1020, 927, 750, 710, 668 cm⁻¹; ¹H NMR (D₂O, 400 MHz, 25° C.): δ=5.05 (dd, J=9.2, 2.3 Hz, 2H), ³¹P NMR (D₂O, 162 MHz, 25° C.): δ=−0.75;

### Dibutyl-2,3-bis(dibenzylphospho)-L-tartrate

To a solution of dibutyl-L-tartrate (460 mg, 1.75 mmol, 1 eq) in dry DCM (10 mL) at room temperature was added tetrazole (0.45 M in acetonitrile) (11.7 mL, 5.26 mmol, 3 eq), followed by dibenzyl N,N-diisopropylphosphoramidite (1.73 mL, 5.26 mmol, 3 eq). The reaction was stirred for 24 h at room temperature, and then cooled to −40° C. 3-chloroperbenzoic acid (969 mg, 5.61 mmol, 3.2 eq) was added portion wise and the reaction was allowed to stir from −40° C. to room temperature over a period of 12 h. Then the reaction mixture was diluted with EtOAc, washed with 10% Na₂SO₃, saturated NaHCO₃, brine then dried over Na₂SO₄ and concentrated in vacuo. The obtained residue was purified by silica gel column chromatography (EtOAc/n-heptane, 5:95 to 50:50) to obtain dibutyl-2,3-bis(dibenzylphospho)-L-tartrate (1.16 g, 84%).

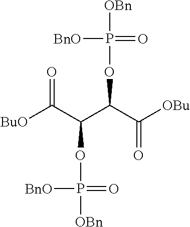

TLC (SiO₂): R_{f}=0.2 (EtOAc/n-heptane, 30:70); ¹H NMR (CDCl₃, 400 MHz, 25° C.): δ=¹H NMR (CDCl₃, 400 MHz, 25° C.): δ=7.39-7.30 (m, 20H), 5.48 (dd, ³J_{HP}=8.6 Hz, ⁴J_{HP}=3.0 Hz, 2H), 5.25 (d of AB, J=11.7 Hz, ³J_{HP}=7.3 Hz, 2H), 5.19 (d of AB, J=11.7 Hz, ³J_{HP}=6.9 Hz, 2H), 4.15 (dt, J=10.6, 7.0 Hz, 2H), 3.96 (dt, J=10.6, 6.8 Hz, 2H), 1.58-1.50 (m, 4H), 1.28 (sextet, J=7.4 Hz, 4H), 0.85 (t, J=7.4 Hz, 6H); ¹³C NMR (CDCl₃, 100 MHz, 25° C.): δ=166.2 (d, ³J_{CP}=1.7 Hz, 2C), 135.7 (d, ³J_{CP}=8.1 Hz, 2C), 135.4 (d, ³J_{CP}=8.1 Hz, 2C), 128.42, 128.37, 127.9, 127.8, 75.4 (dd, ²J_{CP}=7.1 Hz, ³J_{CP}=5.0 Hz, 2C), 69.8 (d, ²J_{CP}=5.6 Hz, 2C), 69.6 (d, ²J_{CP}=6.0 Hz, 2C), 66.5, 30.2, 18.8, 13.5; ³¹P NMR (CDCl₃, 162 MHz, 25° C.): δ=−2.03; HRMS (ESI): m/z: calcd for C₄₀H₄₈LiO₁₂P₂: 789.2776 [M+Li]⁺. found: 789.2829.

### Dibutyl-2,3-bisphospho-L-tartrate tetrasodium salt

To a solution of dibutyl-2,3-bis(dibenzylphospho)-L-tartrate (1.1 g, 1.4 mmol, 1 eq) in EtOH:H₂O (1:1, 40 mL) was added 10% Pd/C (415 mg) and sodium bicarbonate (472 mg, 5.6 mmol, 4 eq). The reaction mixture was stirred at room temperature under an atmosphere of hydrogen for 12 h. The catalyst was filtered through a LCR/PTFE hydrophilic membrane, washed with 2×20 mL of EtOH:H₂O (1:1). The solvent was evaporated under reduced pressure and dried under high vacuum afforded dibutyl-2,3-bisphospho-L-tartrate tetrasodium salt as a white solid (667 mg, 94%).

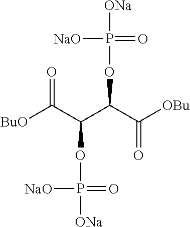

[α]²⁰_{D} 1.0 (c 1.0, H₂O); IR: ν=3572, 2959, 1743, 1727, 1639, 1620, 1612, 1120, 919 cm⁻¹; ¹H NMR (D₂O, 400 MHz, 25° C.): δ=4.79-4.76 (2H, obscured), 4.28 (dt, J=10.6, 7.04 Hz, 2H), 4.13 (dt, J=10.6, 6.96 Hz, 2H), 1.72-1.65 (m, 4H), 1.40 (sextet, J=7.4 Hz, 4H), 0.93 (t, J=7.4 Hz, 6H); ¹³C NMR (D₂O, 100 MHz, 25° C.): δ=171.8 (d, ³J_{CP}=1.1 Hz, 2C), 73.8 (t, J_{CP}=5.3 Hz, 2C), 66.6, 29.8, 18.5, 13.1; ³¹P NMR (D₂O, 162 MHz, 25° C.): δ=3.24; HRMS (ESI): m/z: calcd for C₁₂H₂₁Na₄O₁₂P₂: 511.0094 [M+H]⁺. found: 511.0131.

### Dibutyl-2,3-bisphospho-L-tartrate ditriethylammonium salt

Dibutyl-2,3-bisphospho-L-tartrate tetrasodium salt (400 mg, 0.78 mmol, 1 eq) was dissolved in deionised water (4 mL). This solution was then passed through the column containing a prewashed Dowex 50WX8-200 ion exchange resin (10 g) and the column was eluted with water (4×4 mL). To the combined acidic fractions, triethylamine (0.87 mL, 6.28 mol, 8 eq) was added at room temperature and the mixture was stirred vigorously for 15 minutes. Then the solvent was evaporated on a rotary evaporator and the residue was dried under high vacuum for 1 hr at room temperature to give the dibutyl-2,3-bisphospho-L-tartrate ditriethylammonium salt as a colourless oil.

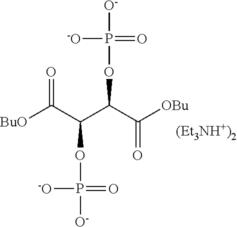

¹H NMR (D₂O, 400 MHz, 25° C.): δ=4.96 (dd, ³J_{HP}=8.9 Hz, ⁴J_{HP}=2.5 Hz, 2H), 4.27 (dt, J=10.6, 6.8 Hz, 2H), 4.14 (dt, J=10.6, 6.8 Hz, 2H), 3.18 (q, J=7.3 Hz, 12H), 1.66 (pent, J=7.4 Hz, 4H), 1.38 (sextet, J=7.4 Hz, 4H), 1.26 (t, J=7.3 Hz, 18H), 0.90 (t, J=7.4 Hz, 6H); ¹³C NMR (D₂O, 100 MHz, 25° C.): δ=169.9 (d, ³J_{CP}=1.4 Hz, 2C), 74.3 (dd, ²J_{CP}=6.1 Hz, ³J_{CP}=5.4 Hz, 2C), 66.7, 46.6, 29.7, 18.4, 13.0, 8.2; ³¹P NMR (D₂O, 162 MHz, 25° C.): δ=−0.63.

### Dibutyl-cyclo-2,3-bisphospho-L-tartrate ditriethylammonium salt

To this dibutyl-2,3-bisphospho-L-tartrate ditriethylammonium salt dissolved in water (8 mL), dicyclohexylcarbodiimide (324 mg, 1.57 mmol, 2.0 eq) dissolved in acetonitrile (16 mL) was added and the mixture was refluxed at 120° C. for 8 h. The mixture was cooled to room temperature and the dicyclohexylurea formed was filtered through a sintered funnel and washed with water (2×4 mL). The filtrate was evaporated on a rotary evaporator and dried under high vacuum at room temperature. The resulting syrupy residue was redissolved in 5 mL of water to remove all dicyclohexylurea that had remained dissolved in acetonitrile, filtered through a sintered funnel, and washed with water (2×4 mL). The filtrate was evaporated on a rotary evaporator and dried under high vacuum at room temperature affording dibutyl-cyclo-2,3-bisphospho-L-tartrate ditriethylammonium salt as a colourless oil.

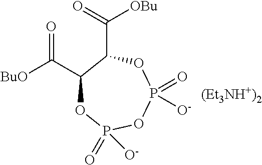

¹H NMR (D₂O, 400 MHz, 25° C.): δ=5.01 (t, J=3.5 Hz, 2H), 4.29 (dt, J=10.7, 6.5 Hz, 2H), 4.18 (dt, J=10.7, 6.5 Hz, 2H), 3.20 (q, J=7.3 Hz, 12H), 1.66 (pent, J=7.4 Hz, 4H), 1.37 (sextet, J=7.4 Hz, 4H), 1.28 (t, J=7.3 Hz, 18H), 0.91 (t, J=7.4 Hz, 6H); ¹³C NMR (D₂O, 100 MHz, 25° C.): δ=167.9 (t, J=8.7 Hz, 2C), 76.7 (t, J=4.5 Hz, 2C), 67.2, 46.6, 29.7, 18.5, 12.9, 8.2; ³¹P NMR (D₂O, 162 MHz, 25° C.): δ=−12.25.

### Dibutyl-cyclo-2,3-bisphospho-L-tartrate disodium salt

Dibutyl-cyclo-2,3-bisphospho-L-tartrate ditriethylammonium salt was dissolved in 4 mL of water, passed through a column containing prewashed Dowex 50WX8-200 (5 g) ion exchange resin and eluted with water (4×4 mL). The pH of the combined acidic fractions was immediately adjusted to around 6.95 with 1N NaOH. Finally, the solvent was evaporated on a rotary evaporator and dried under high vacuum at room temperature to yield the dibutyl-cyclo-2,3-bisphospho-L-tartrate disodium salt (224 mg, 92%) as a white solid.

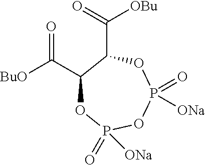

[α]²⁰D 18.6 (c 1.0, H₂O); ¹H NMR (D₂O, 400 MHz, 25° C.): δ=4.96 (dd, ³J_{HP}=5.2 Hz, ⁴J_{HP}=3.1 Hz, 2H), 4.30 (dt, J=10.8, 6.7 Hz, 2H), 4.19 (dt, J=10.8, 6.5 Hz, 2H), 1.67 (pent, J=7.4 Hz, 4H), 1.38 (sextet, J=7.4 Hz, 4H), 0.92 (t, J=7.4 Hz, 6H); ¹³C NMR (D₂O, 100 MHz, 25° C.): δ=167.8 (t, J=5.6 Hz, 2C), 76.7 (t, J=2.9 Hz, 2C), 67.2, 29.6, 18.4, 12.9; ³¹P NMR (D₂O, 162 MHz, 25° C.): δ=−12.10 (s); HRMS (ESI): m/z: calcd for C₁₂H₂₀Li₁O₁₁P₂: 409.0636 [M+Li-2Na]⁻. found: 409.0629.

### Dibenzyl-2,3-bis(dibenzylphospho)-L-tartrate

To a solution of dibenzyl-L-tartrate (760 mg, 2.3 mmol, 1 eq) in dry DCM (10 mL) at room temperature was added tetrazole (0.45 M in acetonitrile) (15.3 mL, 6.9 mmol, 3.0 eq), followed by dibenzyl N,N-diisopropylphosphoramidite (2.27 mL, 6.9 mmol, 3 eq). After being stirred for 12 h at room temperature, the reaction mixture was cooled to −40° C., then 3-chloroperbenzoic acid (1.27 g, 7.36 mmol, 3.2 eq) was added portion wise and the reaction was allowed to stir from −40° C. to room temperature over a period of 12 h. Then the reaction mixture was diluted with EtOAc, washed with 10% Na₂SO₃, saturated NaHCO₃, brine then dried over Na₂SO₄ and concentrated in vacuo. The crude compound was purified by silica gel column chromatography (EtOAc/n-heptane, 5:95 to 35:65) to obtain dibenzyl-2,3-bis(dibenzylphospho)-L-tartrate (1.38 g, 71%).

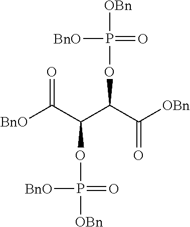 [SMILES: O=C(OCc1ccccc1)[C@H](OP(=O)(OCc1ccccc1)OCc1ccccc1)[C@@H](OP(=O)(OCc1ccccc1)OCc1ccccc1)C(=O)OCc1ccccc1]

TLC (SiO₂): R_{f}=0.24 (EtOAc/n-heptane, 30:70); ¹H NMR (CDCl₃, 400 MHz, 25° C.): δ=7.30-7.27 (m, 30H), 5.47 (dd, ³J_{HP}=8.7 Hz, ⁴J_{HP}=2.9 Hz, 2H), 5.15 (AB, J=11.9 Hz, 4H), 5.12 (dd, J=7.0, 3.7 Hz, 4H), 4.97 (dd, J=6.9, 1.2 Hz, 4H), 4.92 (AB, J=11.9 Hz, 4H); ¹³C NMR (CDCl₃, 100 MHz, 25° C.): δ=166.0 (d, ³J_{CP}=2.1 Hz, 2C), 135.6 (d, ³J_{CP}=7.0 Hz, 2C), 135.4 (d, ³J_{CP}=8.0 Hz, 2C), 134.2, 128.55, 128.53, 128.43, 128.40, 128.37, 75.3 (dd, ²J_{CP}=6.7 Hz, ³J_{CP}=5.2 Hz, 2C), 69.8 (d, ²J_{CP}=5.6 Hz, 2C), 69.6 (d, ²J_{CP}=5.8 Hz, 2C), 68.3; ³¹P NMR (CDCl₃, 162 MHz, 25° C.): δ=−2.02.

### 2,3-Bisphospho-L-tartrate hexasodium salt

To a solution of dibenzyl-2,3-bis(dibenzylphospho)-L-tartrate (168 mg, 0.19 mmol, 1 eq) in EtOH:H₂O (1:1, 10 mL) was added 10% Pd/C (100 mg) and sodium bicarbonate (99 mg, 1.18 mmol, 6 eq). The reaction mixture was stirred at room temperature under an atmosphere of hydrogen for 12 h. The catalyst was filtered through a LCR/PTFE hydrophilic membrane, washed with 2×10 mL of EtOH:H₂O (1:1). The solvent was evaporated under reduced pressure and dried under high vacuum to obtain 2,3-bisphospho-L-tartrate hexasodium salt (84 mg, 97%) as a white solid.

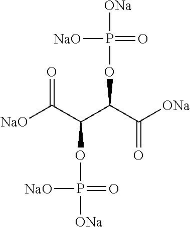

IR: ν=3269, 1602, 1401, 1105, 974, 912, 768, 697 cm⁻¹; ¹H NMR (D₂O, 400 MHz, 25° C.): δ=4.58 (dd, ³J_{HP}=7.6 Hz, ⁴J_{HP}=2.6 Hz, 2H); ³¹P NMR (D₂O, 162 MHz, 25° C.): δ=3.24; HRMS (ESI): m/z: calcd for C₄H₄Na₃O₁₂P₂: 374.8866 [M−H]⁻. found: 374.8883.

### Dimethyl-2,3,4,5-tetrakis(dibenzylphospho)-meso-galactarate

To dimethyl meso-galactarate (600 mg, 2.52 mmol, 1 eq) in DMF (10 mL), tetrazole (0.45 M in acetonitrile) (33.6 mL, 15.2 mmol, 6 eq) at room temperature was added followed by dibenzyl N,N-diisopropylphosphoramidite (4.97 mL, 15.1 mmol, 6 eq). After being stirred for 48 h at room temperature, the reaction mixture was cooled to −40° C., then 3-chloroperbenzoic acid (3.91 g, 22.8 mmol, 9.0 eq) in DCM (40 mL) was added slowly and the reaction was allowed to stir from −40° C. to room temperature over a period of 12 h. Then the reaction mixture was diluted with EtOAc, washed with 10% Na₂SO₃, saturated NaHCO₃, brine then dried over Na₂SO₄ and concentrated in vacuo. The crude compound was purified by silica gel column chromatography (MeOH/DCM, 0.5:99.5 to 3:97) to obtain little impure product. This impure compound was dissolved in EtOAc and precipitated by addition of n-heptane. The suspension was cooled to 0° C., filtered, washed EtOAc:n-heptane (30:70). The resulting white solid was collected and dried under vacuum to obtain pure dimethyl-2,3,4,5-tetrakis(dibenzylphospho)-meso-galactarate (2.57 g, 79%).

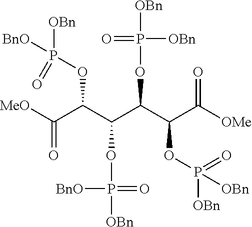 [SMILES: COC(=O)[C@@H](OP(=O)(OCc1ccccc1)OCc1ccccc1)[C@H](OP(=O)(OCc1ccccc1)OCc1ccccc1)[C@H](OP(=O)(OCc1ccccc1)OCc1ccccc1)[C@@H](OP(=O)(OCc1ccccc1)OCc1ccccc1)C(=O)OC]

TLC (SiO₂): R_{f}=0.22 (EtOAc/n-heptane, 70:30); IR: ν=3033, 2955, 1765, 1497, 1455, 1382, 1282, 1216, 1133, 1012, 998, 893, 735, 696 cm⁻¹; ¹H NMR (CDCl₃, 400 MHz, 25° C.): δ=7.35-7.20 (m, 40H), 5.47 (d, J=7.0 Hz, 2H), 5.30-5.13 (m, 14H), 5.03 (dd, J=11.9, 6.5 Hz, 2H), 4.99 (dd, J=11.9, 7.4 Hz, 2H), 3.75 (s, 6H); ¹³C NMR (CDCl₃, 100 MHz, 25° C.): δ=167.7 (s, 2C), 135.9 (d, ³J_{CP}=8.1 Hz, 2C), 135.7 (d, ³J_{CP}=7.5 Hz, 2C), 135.57 (d, ³J_{CP}=6.4 Hz, 2C), 135.50 (d, ³J_{CP}=9.0 Hz, 2C), 128.3, 128.2, 128.0, 127.84, 127.78, 75.2 (m, 2C), 73.6 (brs, 2C), 70.0 (d, ²J_{CP}=5.6 Hz, 2C), 69.79 (d, ²J_{CP}=5.9 Hz, 2C), 69.71 (d, ²J_{CP}=5.7 Hz, 2 C), 69.65 (d, ²J_{CP}=5.5 Hz, 2C), 53.0 (s, 2C); ³¹P NMR (CDCl₃, 162 MHz, 25° C.): δ=−1.96, −2.02.

### Tetrasodium dimethyl-meso-galactarate-2,3,4,5-tetrakisphosphate

To a solution of dimethyl-2,3,4,5-tetrakis(dibenzylphospho)-meso-galactarate (800 mg, 0.62 mmol, 1 eq) in EtOH:H₂O (1:1, 30 mL) was added 10% Pd/C (550 mg) and sodium bicarbonate (210 mg, 2.50 mmol, 4 eq). The solution was evacuated for few minutes, and then the reaction mixture was stirred at room temperature under an atmosphere of hydrogen for 7 h. The catalyst was filtered through a LCR/PTFE hydrophilic membrane, washed with 2×20 mL of EtOH:H₂O (1:1). The solvent was evaporated under reduced pressure and lyophilisation afforded tetrasodium dimethyl-meso-galactarate-2,3,4,5-tetrakisphosphate as a white solid (396 mg, 98%).

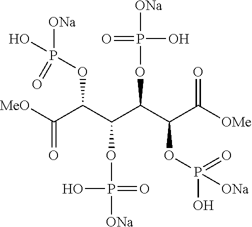

IR: ν=1734, 1209, 1063, 928, 865, 796, 755, 668 cm⁻¹; ¹H NMR (D₂O, 400 MHz, 25° C.): δ=4.96 (d, J=8.1 Hz, 2H), 4.72 (d, J=8.2 Hz, 2H), 3.80 (s, 6H); ¹³C NMR (D₂O, 100 MHz, 25° C.): 171.8, 74.0 (dt, J_{CP}=7.6, 6.1 Hz, 2C), 72.6 (d, J_{CP}=4.9 Hz, 2C), 53.0 (s, 2C); ³¹P NMR (D₂O, 162 MHz, 25° C.): δ=0.05, −1.60.

### Tetrasodium dimethyl-meso-galactarate bispyrophosphates

To a solution of dimethyl-2,3,4,5-tetrakis(dibenzylphospho)-meso-galactarate (1.0 g, 0.78 mmol, 1 eq) in EtOH:H₂O (1:1, 30 mL) was added 10% Pd/C (460 mg) and sodium bicarbonate (262 mg, 3.12 mmol, 4 eq). The reaction mixture was stirred at room temperature under an atmosphere of hydrogen for 24 h. The catalyst was filtered through a LCR/PTFE hydrophilic membrane, washed with 3×10 mL of EtOH:H₂O (1:1). The solvent was evaporated under reduced pressure and dried under high vacuum. The residue was dissolved in water (4 mL) and the solution was passed through a prewashed Dowex H⁺ (5 g) column. The column was eluted with 4×4 mL of water. To the combined acidic fractions, 1N NaOH solution was added until the pH of the solution was 6.86. Then the solvent was evaporated under reduced pressure and dried under high vacuum to obtain sodium salt of dimethyl-meso-galactarate-2,3,4,5-tetrakisphospate (488 mg, 97%) as a white solid. This salt (300 mg, 0.40 mmol, 1 eq) was dissolved in water (4 mL) and the solution was passed through a prewashed Dowex H⁺ (10 g) column. The column was eluted with 4×4 mL of water. The acidic fractions were pooled and then Et₃N (0.90 mL, 6.54 mmol, 16 eq) was added at room temperature. After stirring for 15 minutes, the solvent was evaporated under reduced pressure and dried under high vacuum to obtain hexatriethylammonium salt of dimethyl-meso-galactarate-2,3,4,5-tetrakisphospate as a pale yellow liquid.

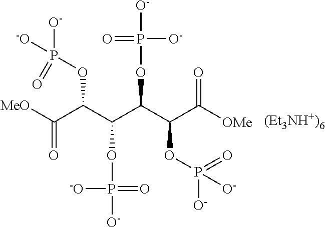

¹H NMR (D₂O, 400 MHz, 25° C.): δ=4.96 (d, J=8.4 Hz, 2H), 4.72 (d, J=7.5 Hz, 2H), 3.80 (s, 6H), 3.21 (q, J=7.3 Hz, 36H), 1.28 (t, J=7.3 Hz, 54H); ³¹P NMR (D₂O, 162 MHz, 25° C.): δ=0.05, −1.64.

This yellow liquid was dissolved in CH₃CN:H₂O (2:1, 24 mL) and heated with dicyclohexylcrbodiimide (330 mg, 1.6 mmol, 4 eq) for 12 h. Then four more equivalents of dicyclohexylcarbodiimide (330 mg, 1.6 mmol, 4 eq) dissolved in acetonitrile (4 mL) was added and refluxed for further 12 h. Again four more equivalents of dicyclohexylcarbodiimide (330 mg, 1.6 mmol, 4 eq) dissolved in acetonitrile (4 mL) was added and refluxed for further 12 h. Then the reaction mixture was cooled to room temperature and the dicyclohexylurea formed was filtered through a sintered funnel and washed with water (3×5 mL). The filtrate was evaporated on a rotary evaporator (60° C., 68-22 mbar) and dried under high vacuum at room temperature. The resulting sticky syrupy residue was redissolved in 5 mL of water to remove all dicyclohexylcarbodiimide that had remained dissolved in acetonitrile, filtered through a sintered funnel, and washed with water (2×5 mL). The filtrate was evaporated on a rotary evaporator (60° C., 68-22 mbar) and dried under high vacuum at room temperature affording a mixture of tetratriethylammonium dimethyl-meso-galactarate bispyrophosphates as a pale yellow liquid. This mixture of tetratriethylammonium dimethyl-meso-galactarate bispyrophosphates were dissolved in mL of water and treated with prewashed Dowex marathon C (Na⁺ form). After stirring for 3 hrs the solution was filtered and washed with water (2×5 mL). To the filtrate again a fresh prewashed Dowex marathon C (Na⁺ form) was added, stirred for 3 hrs and filtered. The above process was repeated until all the triethylammonium ions are exchanged with sodium ions. Finally, the solvent was evaporated on a rotary evaporator (60° C., 68-22 mbar) and dried under high vacuum at room temperature to yield a mixture of tetrasodium dimethyl-meso-galactarate bispyrophosphates (209 mg, 84%) as a pale yellow solid.

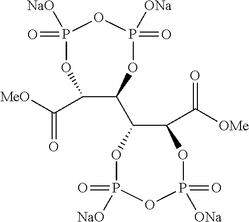

¹H NMR (D₂O, 400 MHz, 25° C.): δ=5.12-4.88 (m, global 4H), 3.83-3.79 (m, global 6H); ³¹P NMR (D₂O, 162 MHz, 25° C.): δ=0.63 to −1.66 (many singlets), −10.48 to −10.94 (many doublets), −10.48 (AB, J=24.6 Hz, 2P), −12.28 to −12.74 (many doublets), −13.29 (AB, J=24.6 Hz, 2P).

### 1,2,3,4-Tetrakis(dibenzylphospho)-meso-erythritol

To a solution of meso-erythritol (368 mg, 3.01 mmol, 1 eq) in dry DMF (20 mL) at room temperature was added tetrazole (0.45 M in acetonitrile) (40.1 mL, 18.0 mmol, 6 eq), followed by dibenzyl N,N-diisopropylphosphoramidite (5.94 mL, 18.0 mmol, 6 eq). After being stirred for 24 h at room temperature, the reaction mixture was cooled to −40° C., then 3-chloroperbenzoic acid (3.3 g, 19.2 mmol, 6.4 eq) in DMF (10 mL) was added slowly and the reaction was allowed to stir from −40° C. to room temperature over a period of 12 h. Then the reaction mixture was diluted with EtOAc, washed with 10% Na₂SO₃, saturated NaHCO₃, brine then dried over Na₂SO₄ and concentrated in vacuo. The obtained residue was purified by silica gel column chromatography (EtOAc/n-heptane, 20:80 to 90:10) to obtain 1,2,3,4-tetrakis(dibenzylphospho)-meso-erythritol (2.2 g, 63%).

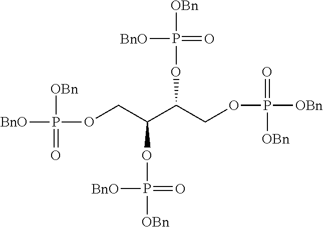 [SMILES: O=P(OCc1ccccc1)(OCc1ccccc1)OC[C@H](OP(=O)(OCc1ccccc1)OCc1ccccc1)[C@@H](COP(=O)(OCc1ccccc1)OCc1ccccc1)OP(=O)(OCc1ccccc1)OCc1ccccc1]

TLC (SiO₂): R_{f}=0.25 (EtOAc/n-heptane, 60:30); IR: ν=3033, 1497, 1455, 1381, 1277, 1215, 998, 880, 736, 696 μm⁻¹; ¹H NMR (CDCl₃, 400 MHz, 25° C.): δ=7.29-7.21 (m, 40H), 5.02-4.97 (m, 16H), 4.79-4.72 (m, 2H), 4.32-4.25 (m, 2H), 4.14 (dt, J=11.4, 4.8 Hz, 2H); ¹³C NMR (CDCl₃, 100 MHz, 25° C.): 135.6 (t, ³J_{CP}=6.0 Hz), 128.59, 128.56, 128.52, 128.03, 127.97, 127.96, 75.1 (dd, J_{CP}=14.3, 6.0 Hz), 69.7 (dd, J_{CP}=5.5, 3.7 Hz), 69.6 (d, J_{CP}=5.5 Hz), 65.4 (d, J_{CP}=3.6 Hz); ³¹P NMR (CDCl₃, 162 MHz, 25° C.): δ=−1.03, −1.60; HRMS (ESI): m/z: calcd for C₆₀H₆₂Na₁O₁₆P₄: 1185.2881 [M+Na]⁺. found: 1185.2811.

### 1,2,3,4-Tetrakisphospho-meso-erythritol tetrasodium salt

To a solution of 1,2,3,4-tetrakis(dibenzylphospho)-meso-erythritol (445 mg, 0.38 mmol, 1 eq) in EtOH:H₂O (1:1, 10 mL) was added 10% Pd/C (350 mg). The solution was evacuated for few minutes, and then the reaction mixture was stirred at room temperature under an atmosphere of hydrogen for 12 h. The catalyst was filtered through a LCR/PTFE hydrophilic membrane, washed with 2×20 mL of EtOH:H₂O (1:1). The solvent was evaporated under reduced pressure and dried under high vacuum afforded 1,2,3,4-tetrakisphospho-meso-erythritol tetrasodium salt (196 mg, 97%) as a white solid.

### Tetrasodium meso-erythritol bispyrophosphate

To a solution of 1,2,3,4-tetrakis(dibenzylphospho)-meso-erythritol (458 mg, 0.39 mmol, 1 eq) in EtOH:H₂O (1:1, 20 mL) was added 10% Pd/C (360 mg) followed by solid NaHCO₃ (132 mg, 1.57 mmol, 4 eq). The reaction mixture was stirred at room temperature under an atmosphere of hydrogen for 12 h. The catalyst was filtered through a LCR/PTFE hydrophilic membrane, washed with 2×20 mL of EtOH:H₂O (1:1). The solvent was evaporated under reduced pressure and dried under high vacuum afforded 1,2,3,4-tetrakisphospho-meso-erythritol tetrasodium salt in quantitative yield. The residue was dissolved in water (4 mL) and the solution was passed through a prewashed Dowex H⁺ column. The column was eluted with 3×2 mL of water. The acidic fractions were pooled and then Et₃N (0.87 mL, 6.24 mmol, 16 eq) was added at room temperature. After stirring for 15 minutes, the solvent was evaporated under reduced pressure and dried under high vacuum to obtain tetratriethylammonium meso-erythritol 1,2,3,4-tetrakisphosphate as a pale yellow liquid.

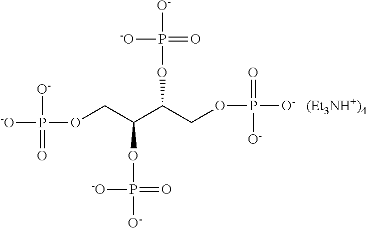

¹H NMR (D₂O, 400 MHz, 25° C.): δ=4.46-4.41 (m, 2H), 4.20-4.13 (m, 2H), 4.11-4.13 (m, 2H), 3.19 (q, J=7.3 Hz, 24H), 1.27 (t, J=7.3 Hz, 36H); ¹³C NMR (D₂O, 100 MHz, 25° C.): δ=73.1 (dt, J_{CP}=7.7, 6.1 Hz, 2C), 64.1 (d, J_{CP}=3.8 Hz, 2C), 46.6 (s), 8.2 (s); ³¹P NMR (D₂O, 162 MHz, 25° C.): δ=1.06, −0.59.

This yellow liquid was dissolved in CH₃CN:H₂O (2:1, 24 mL) and heated with DCC (321 mg, 1.56 mmol, 4 eq) for 8 h. The mixture was cooled to room temperature and the dicyclohexylurea formed was filtered through a sintered funnel and washed with water (3×4 mL). The filtrate was evaporated on a rotary evaporator (60° C., 68-22 mbar) and dried under high vacuum at room temperature. The resulting sticky syrupy residue was redissolved in 4 mL of water to remove all dicyclohexylcarbodiimide that had remained dissolved in acetonitrile, filtered through a sintered funnel, and washed with water (2×4 mL). The filtrate was evaporated on a rotary evaporator (60° C., 68-22 mbar) and dried under high vacuum at room temperature affording a mixture of tetratriethylammonium meso-erythritol bispyrophosphate as a pale yellow liquid.

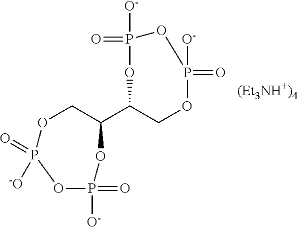

¹H NMR (D₂O, 400 MHz, 25° C.): δ=4.54-4.38 (m, global 2.5H), 4.33-4.10 (m, global 3.5H); ³¹P NMR (D₂O, 162 MHz, 25° C.): δ=−9.72 (AB, J=18.1 Hz, 2P, major isomer), −10.77 (AB, J=24.9 Hz, 2P, minor isomer), −11.22 (AB, J=18.1 Hz, 2P, major isomer), −11.93 (AB, J=24.9 Hz, 2P, minor isomer).

This tetratriethylammonium meso-erythritol bispyrophosphate salt was dissolved in 4 mL of water, passed through a column containing prewashed Dowex 50×8-200 H⁺ form (5 g) ion exchange resin and eluted with water (4×2 mL). The combined acidic fractions were immediately adjusted to pH 6.8 with 1N NaOH solution at room temperature. Finally, the solvent was evaporated on a rotary evaporator (60° C., 68-22 mbar) and dried under high vacuum at room temperature to yield a mixture (66.6% of major isomer and 33.3% of minor isomer) of tetrasodium meso-erythritol bispyrophosphate (170 mg, 87%) as a pale yellow solid.

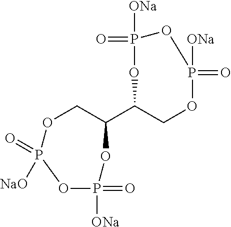

¹H NMR (D₂O, 400 MHz, 25° C.): δ=4.58-4.42 (m, global 2.5H), 4.36-4.15 (m, global 3.5H); ³¹P NMR (D₂O, 162 MHz, 25° C.): δ=−9.57 (AB, J=18.0 Hz, 2P, major isomer), −10.56 (AB, J=24.8 Hz, 2P, minor isomer), −11.06 (AB, J=18.0 Hz, 2P, major isomer), −11.73 (AB, J=24.8 Hz, 2P, minor isomer).

### 1,3,4,5-Tetrakis(dibenzylphospho)pentaerythritol

To a solution of pentaerythritol (272 mg, 2.0 mmol, 1 eq) in dry DCM (40 mL) at room temperature was added tetrazole (0.45 M in acetonitrile) (26.6 mL, 12.0 mmol, 6 eq), followed by dibenzyl N,N-diisopropylphosphoramidite (3.94 mL, 12.0 mmol, 6 eq). After being stirred for 48 h at room temperature, the reaction mixture was cooled to −40° C., then 3-chloroperbenzoic acid (3.1 g, 18.0 mmol, 9 eq) was added portion wise and the reaction was allowed to stir from −40° C. to room temperature over a period of 9 h. Then the reaction mixture was diluted with EtOAc, washed with 10% Na₂SO₃, saturated NaHCO₃, brine then dried over Na₂SO₄ and concentrated in vacuo. The obtained residue was purified by silica gel column chromatography (EtOAc/n-heptane, 20:80 to 80:20) to obtain 1,3,4,5-tetrakis(dibenzylphospho)pentaerythritol (1.95 g, 82%) as a white solid.

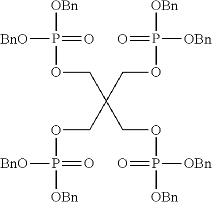 [SMILES: O=P(OCc1ccccc1)(OCc1ccccc1)OCC(COP(=O)(OCc1ccccc1)OCc1ccccc1)(COP(=O)(OCc1ccccc1)OCc1ccccc1)COP(=O)(OCc1ccccc1)OCc1ccccc1]

TLC (SiO₂): R_{f}=0.25 (EtOAc/n-heptane, 80:20); ¹H NMR (CDCl₃, 400 MHz, 25° C.): δ=7.28-7.21 (brs, 40H), 5.02-4.97 (m, 16H), 4.97 (d, J=11.9 Hz, 8H), 4.92 (d, J=11.9 Hz, 8H), 3.87 (d, J=4.1 Hz, 8H); ¹³C NMR (CDCl₃, 100 MHz, 25° C.): 135.5 (brs), 135.43 (brs), 128.5, 127.9, 69.44 (brs, 4C), 69.39 (brs, 4C), 64.3 (d, ²J_{CP}=17.5 Hz, 4C), 44.6 (brt, 1C); ³¹P NMR (CDCl₃, 162 MHz, 25° C.): δ=−1.04.

### Tetrasodium pentaerythritol 1,3,4,5-tetrakisphosphate

To a solution of 1,3,4,5-tetrakis(dibenzylphospho)pentaerythritol (950 mg, 0.8 mmol, 1 eq) in EtOH:H₂O (1:1, 40 mL) was added 10% Pd/C (700 mg) and NaHCO₃ (271 mg, 3.23 mmol, 4 eq). The solution was evacuated for few minutes, and then the reaction mixture was stirred at room temperature under an atmosphere of hydrogen for 7 h. The catalyst was filtered through a LCR/PTFE hydrophilic membrane, washed with 2×20 mL of EtOH:H₂O (1:1). The solvent was evaporated under reduced pressure and lyophilisation afforded tetrasodium pentaerythritol 1,3,4,5-tetrakisphosphate as a white solid (432 mg, 96%).

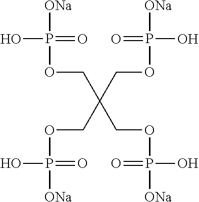

IR: ν=3388, 1181, 1035, 921, 826 cm⁻¹; ¹H NMR (D₂O, 400 MHz, 25° C.): δ=3.90 (bd, J=4.3 Hz, 8H); ¹³C NMR (D₂O, 100 MHz, 25° C.): 62.7 (d, ²J_{CP}=5.0 Hz, 4C), 45.5 (quintet, ³J_{CP}=8.3 Hz, 1C); ³¹P NMR (D₂O, 162 MHz, 25° C.): δ=0.71.

### Tetrasodium pentaerythritol (1,3):(4,5)-bispyrophosphate

To a solution of 1,3,4,5-tetrakis(dibenzylphospho)-pentaerythritol (500 mg, 0.42 mmol, 1 eq) in EtOH:H₂O (1:1, 20 mL) was added 10% Pd/C (360 mg) followed by Et₃N (0.94 mL, 6.80 mmol, 16 eq). The reaction mixture was stirred at room temperature under an atmosphere of hydrogen for 24 h. The catalyst was filtered through a LCR/PTFE hydrophilic membrane, washed with 2×20 mL of EtOH:H₂O (1:1). The solvent was evaporated under reduced pressure and dried under high vacuum afforded tetratriethylammonium pentaerythritol-1,3,4,5-tetrakisphosphate as a pale yellow liquid.

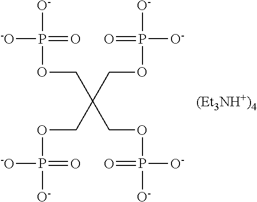

¹H NMR (D₂O, 400 MHz, 25° C.): δ=3.87 (bd, J=4.0 Hz, 8H), 3.18 (q, J=7.3 Hz, 24H), 1.28-1.24 (m, 36H); ³¹P NMR (D₂O, 162 MHz, 25° C.): δ=0.63.

This yellow liquid was dissolved in CH₃CN:H₂O (2:1, 24 mL) and heated with DCC (312 mg, 1.51 mmol, 4 eq) for 12 h. Four more equivalents of dicyclohexylcarbodiimide (312 mg, 1.51 mmol, 4 eq) dissolved in acetonitrile (4 mL) was added and refluxed for further 12 h. The mixture was cooled to room temperature and the dicyclohexylurea formed was filtered through a sintered funnel and washed with water (3×10 mL). The filtrate was evaporated on a rotary evaporator (60° C., 68-22 mbar) and dried under high vacuum at room temperature. The resulting sticky syrupy residue was redissolved in 5 mL of water to remove all dicyclohexylcarbodiimide that had remained dissolved in acetonitrile, filtered through a sintered funnel, and washed with water (2×5 mL). The filtrate was evaporated on a rotary evaporator (60° C., 68-22 mbar) and dried under high vacuum at room temperature affording tetratriethylammonium pentaerythritol-(1,3):(4,5)-bispyrophosphate as a pale yellow liquid.

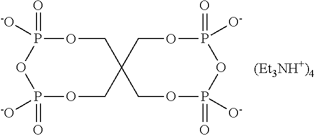

¹H NMR (D₂O, 400 MHz, 25° C.): δ=3.96 (t, J=5.6 Hz, 8H), 3.20 (q, J=7.3 Hz, 24H), 1.28 (t, J=7.3 Hz, 36H); ³¹P NMR (D₂O, 162 MHz, 25° C.): δ=−10.2 (s, 4P).

This tetratriethylammonium pentaerythritol-(1,3):(4,5)-bispyrophosphate salt was dissolved in 10 mL of water and treated with prewashed Dowex Marathon C Sodium form (2 g) ion exchange resin for 2 h. Then the resin was filtered off and washed with water (2×5 mL). To the filtrate again a freshly washed Dowex Marathon C Sodium form (2 g) ion exchange resin was added and stirred for 2 h. Then the resin was filtered off and washed with water (2×5 mL). This process has repeated further 2 times.

Finally, the solvent was evaporated on a rotary evaporator (60° C., 68-22 mbar) and dried under high vacuum at room temperature to yield tetrasodium pentaerythritol-(1,3):(4,5)-bispyrophosphate (186 mg, 86%) as a pale yellow solid.

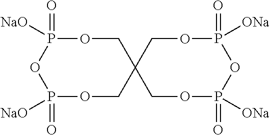

IR: ν=3320, 1258, 1158, 1122, 1086, 1034, 943, 838, 787, 714 cm⁻¹; ¹H NMR (D₂O, 400 MHz, 25° C.): δ=3.98 (dd, J=5.6, 5.2 Hz, 8H); ¹³C NMR (D₂O, 100 MHz, 25° C.): 64.0 (s), 45.5 (t, ³J_{CP}=3.0 Hz, 1C); ³¹P NMR (D₂O, 162 MHz, 25° C.): δ=−10.2 (s, 4P).

### 1,3,4,6-Tetrakis(dibenzylphospho) 2,5-anhydro-D-mannitol

To a solution of 2,5-anhydro-D-mannitol (250 mg, 1.71 mmol, 1 eq) in dry DCM (30 mL) at room temperature was added tetrazole (0.45 M in acetonitrile) (22.8 mL, 10.26 mmol, 6 eq), followed by dibenzyl N,N-diisopropylphosphoramidite (3.37 mL, 10.26 mmol, 6 eq). After being stirred for 48 h at room temperature, the reaction mixture was cooled to −40° C., then 3-chloroperbenzoic acid (2.65 g, 15.39 mmol, 9 eq) in DCM (30 mL) was added slowly and the reaction was allowed to stir from −40° C. to room temperature over a period of 9 h. Then the reaction mixture was diluted with EtOAc, washed with 10% Na₂SO₃, saturated NaHCO₃, brine then dried over Na₂SO₄ and concentrated in vacuo. The obtained residue was purified by silica gel column chromatography (EtOAc/n-heptane, 20:80 to 100:0) to obtain 1,3,4,6-tetrakis(dibenzylphospho) 2,5-anhydro-D-mannitol (1.58 g, 76%) as a white solid.

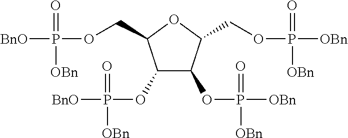 [SMILES: O=P(OCc1ccccc1)(OCc1ccccc1)OC[C@H]1O[C@H](COP(=O)(OCc2ccccc2)OCc2ccccc2)[C@@H](OP(=O)(OCc2ccccc2)OCc2ccccc2)[C@@H]1OP(=O)(OCc1ccccc1)OCc1ccccc1]

TLC (SiO₂): R_{f}=0.23 (EtOAc/n-heptane, 80:20); [α]²⁰_{D} 12.2 (c 1.0, CHCl₃); IR: ν=3033, 1497, 1455, 1381, 1277, 1215, 1007, 998, 884, 737, 696 cm⁻¹; ¹H NMR (CDCl₃, 400 MHz, 25° C.): δ=7.31-7.25 (m, 40H), 5.05-4.98 (m, 18H), 4.19-4.15 (m, 2H), 4.13-4.10 (m, 4H); ¹³C NMR (CDCl₃, 100 MHz, 25° C.): 135.6 (d, ³J_{CP}=7.0 Hz, 4C), 135.2 (d, ³J_{CP}=6.6 Hz, 2C), 135.6 (d, ³J_{CP}=6.5 Hz, 2C), 128.52, 128.48, 128.46, 128.42, 128.36, 127.95, 127.94, 127.82, 127.79, 80.9 (dd, J_{CP}=7.4, 3.8 Hz), 80.7 (t, J_{CP}=6.1 Hz), 69.7 (t, J_{CP}=5.5 Hz), 69.3 (dd, J_{CP}=5.3, 2.9 Hz), 65.7 (d, J_{CP}=5.5 Hz); ³¹P NMR (CDCl₃, 162 MHz, 25° C.): δ=−0.92, −1.78.

### Tetrasodium 2,5-anhydro-D-mannitol 1,3,4,6-tetrakisphosphate

To a solution of 1,3,4,6-tetrakis(dibenzylphospho)-2,5-anhydro-D-mannitol (1.0 g, 0.83 mmol, 1 eq) in EtOH:H₂O (1:1, 40 mL) was added 10% Pd/C (720 mg) and NaHCO₃ (279 mg, 3.3 mmol, 4 eq). The solution was evacuated for few minutes, and then the reaction mixture was stirred at room temperature under an atmosphere of hydrogen for 6 h. The catalyst was filtered through a LCR/PTFE hydrophilic membrane, washed with 2×20 mL of EtOH:H₂O (1:1). The solvent was evaporated under reduced pressure and lyophilisation afforded tetrasodium 2,5-anhydro-D-mannitol-1,3,4,6-tetrakisphosphate as a white solid (466 mg, 98%).

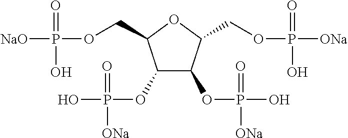

[α]²⁰_{D} 26.5 (c 1.0, H₂O); IR: ν=3399, 1657, 1190, 1046, 919 cm⁻¹; ¹H NMR (D₂O, 400 MHz, 25° C.): δ=4.67 (dd, J_{HP}=2.7, 1.5 Hz, 2H), 4.35-4.32 (m, 2H), 4.10-3.99 (m, 4H); ¹³C NMR (D₂O, 100 MHz, 25° C.): 82.5 (dd, J_{CP}=13.1, 7.9 Hz, 2C), 79.5 (t, J_{CP}=8.1 Hz, 2C), 64.3 (d, ²J_{CP}=8.1 Hz, 2C); ³¹P NMR (D₂O, 162 MHz, 25° C.): δ=0.47, −0.58.

### Tetrasodium 2,5-anhydro-D-mannitol bispyrophosphate

To a solution of 1,3,4,6-tetrakis(dibenzylphospho) 2,5-anhydro-D-mannitol (500 mg, 0.42 mmol, 1 eq) in EtOH:H₂O (1:1, 20 mL) was added 10% Pd/C (360 mg) followed by Et₃N (0.94 mL, 6.80 mmol, 16 eq). The reaction mixture was stirred at room temperature under an atmosphere of hydrogen for 24 h. The catalyst was filtered through a LCR/PTFE hydrophilic membrane, washed with 2×20 mL of EtOH:H₂O (1:1). The solvent was evaporated under reduced pressure and dried under high vacuum afforded tetratriethylammonium 2,5-anhydro-D-mannitol-1,3,4,6-tetrakisphosphate as a pale yellow liquid.

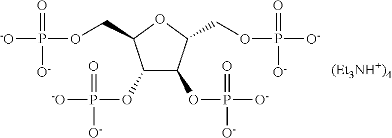

¹H NMR (D₂O, 400 MHz, 25° C.): δ=4.70-4.65 (m, 2H), 4.31-4.29 (m, 2H), 4.01-3.98 (m, 4H), 3.19 (q, J=7.3 Hz, 24H), 1.28 (t, J=7.3 Hz, 36H); ³¹P NMR (D₂O, 162 MHz, 25° C.): δ=0.42, −0.59.

This yellow liquid was dissolved in CH₃CN:H₂O (2:1, 24 mL) and heated with DCC (346 mg, 1.68 mmol, 4 eq) for 12 h. Four more equivalents of dicyclohexylcarbodiimide (346 mg, 1.68 mmol, 4 eq) dissolved in acetonitrile (4 mL) was added and refluxed for further 12 h. The mixture was cooled to room temperature and the dicyclohexylurea formed was filtered through a sintered funnel and washed with water (3×10 mL). The filtrate was evaporated on a rotary evaporator (60° C., 68-22 mbar) and dried under high vacuum at room temperature. The resulting sticky syrupy residue was redissolved in 5 mL of water to remove all dicyclohexylcarbodiimide that had remained dissolved in acetonitrile, filtered through a sintered funnel, and washed with water (2×5 mL). The filtrate was evaporated on a rotary evaporator (60° C., 68-22 mbar) and dried under high vacuum at room temperature affording a mixture of tetratriethylammonium 2,5-anhydro-D-mannitol bispyrophosphates as a pale yellow liquid.

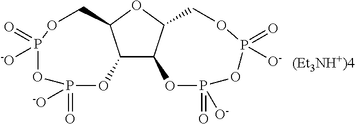

¹H NMR (D₂O, 400 MHz, 25° C.): δ=4.75-4.72 (m, 2H), 4.28-3.90 (m, 6H), 3.16 (q, J=7.3 Hz, 24H), 1.24 (t, J=7.3 Hz, 36H); ³¹P NMR (D₂O, 162 MHz, 25° C.): δ=0.19 (singlet, major isomer), 0.02, −0.83, −0.92 (singlets), −11.23 (singlet, major isomer), −11.93 (broad singlet, minor isomer), −12.71 (broad singlet, minor isomer).

This salt was dissolved in 5 mL of water and treated with prewashed Dowex Marathon C Sodium form (4 g) ion exchange resin for 2 h. Then the resin was filtered off and washed with water (2×5 mL). To the filtrate again a freshly washed Dowex Marathon C Sodium form (4 g) ion exchange resin was added and stirred for 2 h. Then the resin was filtered off and washed with water (2×5 mL). This process has repeated further 2 times.

Finally, the solvent was evaporated on a rotary evaporator (60° C., 68-22 mbar) and dried under high vacuum at room temperature to yield a mixture of tetrasodium 2,5-anhydro-D-mannitol bispyrophosphates (198 mg, 89%) as a pale yellow solid.

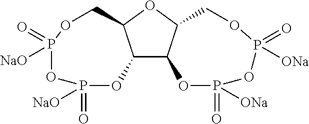

¹H NMR (D₂O, 400 MHz, 25° C.): δ=4.79 (obscured, 2H), 4.33-3.71 (m, 6H); ³¹P NMR (D₂O, 162 MHz, 25° C.): δ=0.70 (singlet, major isomer), 0.31, −0.22, −0.40 (singlets), −10.89 (singlet, major isomer), −11.58 (AB, J=24.2 Hz, minor isomer), −12.38 (AB, J=24.2 Hz, minor isomer).

### Example 2

### Allosteric Modulation of Hemoglobin

In vitro experiments were performed on free hemoglobin and whole blood.

Some of the compounds described herein were tested for P₅₀, the partial pressure of oxygen for half-saturation, on free hemoglobin (Hb) as well as human whole blood (WB) in different concentrations.

The hemoglobin solution was prepared from red blood cells concentrate (EFS-Alsace) by washing three times with 1 volume of saline (1500×g, 10 min), the cells were hemolysed by addition of 2 volumes of cold distilled water. After centrifugation (7000×g, 30 min) for stroma removal, 5 ml of the clear hemoglobin solution were placed on a 2.5 cm×30 cm column of Sephadex G-25 equilibrated with 0.1 M sodium chloride+10⁻⁵ M EDTA. The protein was eluted with the same solution at a rate of about 20 ml/h [Benesch, R.; Benesch, R. E. and Yu, C. I. Reciprocal binding of oxygen and diphosphoglycerate by human hemoglobin. Proc. Natl. Acad. Sci. USA (1968) 59, 526-532].

The allosteric modulation of the effectors was measured by the change in p50, the partial pressure of oxygen for half-saturation. The pH of the compound solutions was adjusted to approximately 7.0. Oxygen equilibrium curves (OEC) were carried out with the Hemox Analyzer (TCS Scientific Co.) under the following conditions: pH 7.4, 135 mM NaCl, 5 mM KCl and 30 mM TES (N—[tris(hydroxymethyl)methyl]-2-aminoethanesulfonic acid) buffer at 37° C. The concentration of free hemoglobin was 5·10⁻³ M (577 nm, ε=58.4 mM⁻¹ cm⁻¹ per tetramer) and the final concentrations of the allosteric effectors were of 1.0·10⁻¹ M, resulting in an effector/Hb ratio of 20.

Human whole blood was freshly withdrawn from healthy donors in heparinized microtubes. The pH of the compound solutions was adjusted to approximately 7.0 and whole blood volumes at 1:1 ratios were incubated individually for 1-2 h at 37° C. with the above compounds. The measurement of the OEC was carried out with the Hemox Analyzer (TCS Scientific Co.) under the following conditions: pH 7.4, 135 mM NaCl, 5 mM KCl and 30 mM TES (N-[tris(hydroxymethyl)methyl]-2-aminoethanesulfonic acid) buffer at 37° C.

TABLE 1
Effect of perphosphorylated polyols on hemoglobin
oxygen affinity in different blood matrices.

| | | | | P₅₀ | | P₅₀ |
|---|---|---|---|---|---|---|
| | Blood | Conc. | | control | P₅₀ | increase |
| Compound | matrix | (mM) | n | (Torr) | (Torr) | (%) |
| Diethyl-L- | Hb | 100 | 3 | 10.90 ± 0.82 | 17.45 ± 0.38 | 60 |
| tartrate | WB | 120 | 3 | 27.04 ± 0.90 | 37.26 ± 1.62 | 38 |
| bisphosphate | | | | | | |
| (SP1-65) | | | | | | |
| Dibutyl-L- | WB | 120 | 3 | 27.04 ± 0.90 | 29.50 ± 2.26 | 9 |
| tartrate | | | | | | |
| bisphosphate | | | | | | |
| (SP1-80) | | | | | | |
| Dibutyl-L- | WB | 60 | 3 | 27.74 ± 1.50 | 30.91 ± 1.76 | 11 |
| tartrate | WB | 120 | 3 | 27.74 ± 1.50 | 30.77 ± 1.44 | 11 |
| bisphosphate | | | | | | |
| (SP1-219) | | | | | | |
| L-tartrate | Hb | 100 | 3 | 10.93 ± 1.05 | 24.76 ± 1.31 | 127 |
| bisphosphate | | | | | | |
| (SP1-90) | | | | | | |
| meso-eryth- | Hb | 100 | 3 | 11.72 ± 0.53 | 29.40 ± 1.20 | 151 |
| ritol tetra- | WB | 120 | 3 | 25.73 ± 0.61 | 30.17 ± 0.94 | 17 |
| phosphate | | | | | | |
| (SP1-134) | | | | | | |

### Example 3

### Inhibition of PI3K Activity

Several compounds were tested for activity against class I PI3K. The assay was conducted using the HTRF Assay Platform (Reaction Biology Corporation (RBC), Malvern, Pa.). In this assay, PIP3 product is detected by displacement of biotin-PIP3 from an energy transfer complex consisting of Europium labeled anti-GST monoclonal antibody, a GST-tagged pleckstrin homology (PH) domain, biotinylated PIP3 and Streptavidin-Allophycocyanin (APC). Excitation of Europium in the complex results in an energy transfer to the APC and a fluorescent emission at 665 nm. The PIP3 product formed by PI3-Kinase(h) activity displaces biotin-PIP3 from the complex resulting in a loss of energy transfer and thus a decrease in signal.

The following enzymes were tested:
Human PI3Kα (p110α/p85α): Complex of N-terminal GST-tagged recombinant full-length human p110a (GenBank Accession No. U79143), and recombinant full length, human p85a (no tag) (GenBank Accession No. XM_{—}043865). Coexpressed in a Baculovirus infected Sf9 cell expression system. p110α MW=155 kDa, p85α MW=83.5 kDa.
Human PI3Kβ (p110β/p85α): Complex of N-terminal 6His-tagged recombinant full-length human p110β (GenBank Accession No. NM_{—}006219), and recombinant full length, human p85α (no tag) (GenBank Accession No. XM_{—}043865). Coexpressed in a Baculovirus infected Sf21 cell expression system. p110β MW=124 kDa, p85α MW=83.7 kDa.
Human PI3Kγ (p120γ): (GenBank Accession No. AF327656), full length with N-terminal His tag, expressed in a Baculovirus infected Sf9 cell expression system. MW=131 kDa.
Human PI3Kδ (p110δ/p85α): Complex of N-terminal GST tagged recombinant full-length human p110δ (GenBank Accession No. NM_{—}005026), and recombinant full length, human p85α (GenBank Accession No. XM_{—}043865). Coexpressed in a Baculovirus infected Sf9 cell expression system. p110δ MW=146 kDa, p85α MW=83.5 kDa.

### Results

As shown in Table 2 below, several compounds showed activity against PI3K. OXY111A showed activity against PI3Kβ and δ, while OXY3008 was a pan inhibitor.

TABLE 2
Effect of compounds on PIK3 activity

| Structure | 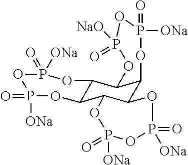 | 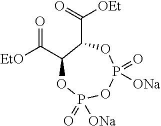 | 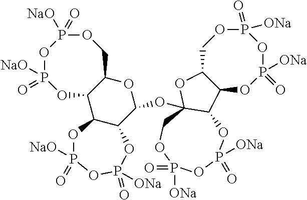 |
|---|---|---|---|
| Number | OXY111A | OXY3012 | OXY3010 |
| Name | Myo-ITPP | L-DET | Sucrose tetra PP |
| Batch | | JRM1-179 | |
| Formula | C6H6Na6O21P6 | C8H12Na2O11P2 | C12H14Na8O31P8 |
| Chemotype or Chemical class | Pyrophosphate polyol | Pyrophosphate polyol | Pyrophosphate polyol |
| Polar Surface Area | 240.87 | 132.89 | 348.85 |
| Salt Weight | 737.88 | 392.10 | 1085.93 |
| Free Weight | | | |
| PI3 Kinase Inhibition | Yes | Yes | |

| Structure | 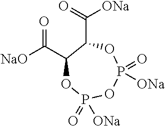 | 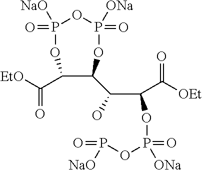 | 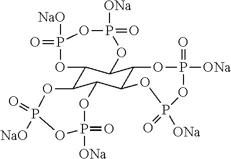 |
|---|---|---|---|
| Number | OXY3011 | OXY3001 | OXY3009 |
| Name | L-NaTartrate | | scyllo-ITPP |
| Batch | JRM1-180 | | |
| Formula | C4H2Na4O11P2 | C10H14Na4O18P4 | C6H6Na6O21P6 |
| Chemotype or Chemical class | Pyrophosphate polyol | Pyrophosphate polyol | Pyrophosphate polyol |
| Polar Surface Area | 132.89 | 213.8 | 251.54 |
| Salt Weight | 379.96 | 638.06 | 737.88 |
| Free Weight | | | |
| PI3 Kinase Inhibition | | | |

| Structure | 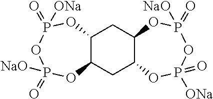 | 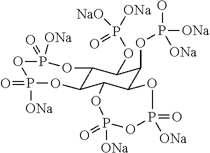 | 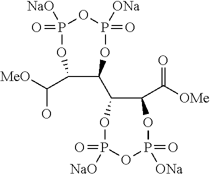 |
|---|---|---|---|
| Number | OXY3008 | OXY3007 | OXY3006 |
| Name | | myo-BPBPP | meso-Me Galactarate |
| Batch | | | JRM1-164 |
| Formula | C6H8Na4O14P4 | C6H6Na8O22P6 | C8H10Na4O18P4 |
| Chemotype or Chemical class | Pyrophosphate polyol | Pyrophosphate polyol | Pyrophosphate polyol |
| Polar Surface Area | 160.58 | 250.1 | 213.18 |
| Salt Weight | 519.97 | 799.86 | 610.01 |
| Free Weight | | | |
| PI3 Kinase Inhibition | Yes | | Yes |

| Structure | 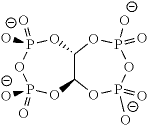 | 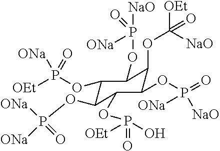 | 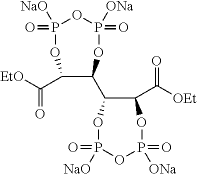 |
|---|---|---|---|
| Number | OXY3005 | OXY3004 | OXY3003 |
| Name | | | Meso-Et Galactarate |
| Batch | | SP1-162 | |
| Formula | C2H2Na4O14P4 | C8H12Na8O22P6 | C10H14Na4O18P4 |
| Chemotype or Chemical class | Pyrophosphate polyol | Pyrophosphate polyol | Pyrophposphate polyol |
| Polar Surface Area | 215.9 | 337.97 | 213.18 |
| Salt Weight | 465.8 | 829.93 | 638.06 |
| Free Weight | 373.84 | | |
| PI3 Kinase Inhibition | | | |

| Structure | 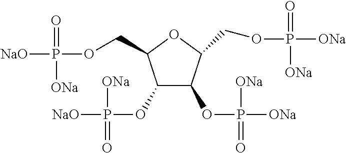 |
|---|---|
| Number | OXY3002 |
| Name | |
| Batch | SP3-30 |
| Formula | C6H8Na8O17P4 |
| Chemotype or Chemical class | Phosphae polyol |
| Polar Surface Area | 188.27 |
| Salt Weight | 659.93 |
| Free Weight | |
| PI3 Kinase Inhibition | |

1. A method for treating a patient having a condition associated with PI3 kinase activity, comprising administering to the patient an effective amount of a PI3K inhibitor having a pyrophosphoryl ring, or structural mimetic thereof.

2. The method of claim 1, wherein the patient's condition is associated with, or characterized by, an amplification, somatic mutation, chromosomal rearrangement, overexpression, or overactivity of a PI3K.

3. The method of claim 2, wherein the PI3K is one or more of a class I PI3K, class II PI3K, class III, and class IV PI3K.

4. The method of any one of claim 1, wherein the patient has a mutation or alteration in a PI3K pathway gene.

5. The method of claim 4, wherein the condition is associated with an inactivation of the tumor suppressor PTEN.

6. The method of claim 1, wherein the condition is an allergic disease, inflammation, heart disease, autoimmunity, diabetes mellitus, or cancer.

7. The method of claim 6, wherein the condition is cancer, and the cancer is one or more of ovarian, cervical, endometrial, colorectal, breast, pancreatic, gastric, glioblastoma, melanoma, liver, prostate, leukemia, lymphoma, head and neck, gastric, kidney, or lung.

8. The method of claim 7, wherein the patient is tested prior to therapy for an alteration, hyperactivity, or overexpression of a PI3K, or mutation or inactivation of PTEN.

9. The method of claim 1, wherein the method comprises administering a compound that is a polyphosphorylated inositol having at least one internal pyrophosphate ring.

10. The compound of claim 9, wherein the compound is myo-inositol 1,6;2,3;4,5 tripyrophosphate, or a pharmaceutically acceptable prodrug or salt thereof.

11. The method of claim 1, wherein the compound is a compound of Formula (II): 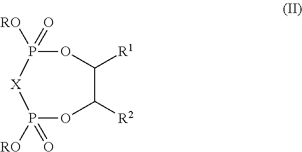
or a pharmaceutically acceptable salt, solvate, and/or prodrug thereof, where:
R is hydrogen, a cation, or an organic moiety;
R1 and R2, taken together, may form a ring of 5 or 6 carbon atoms which is optionally substituted, or alternatively, a heterocyclic 5-, 6-, or 7-membered ring, which is optionally substituted; or
R1 and R2, taken together, form a polyol core selected from an aldose, an alditol, and an aldaric acid, which may be substituted; or
one or both of R1 and R2 are carboxy or an ester thereof of from 1 to 10 carbon atoms; or
one of R1 and R2 form a pyrophosphate ring, which may be further substituted by a carboxy group or ester thereof.

12. The method of claim 1, wherein the patient is treated with one or more additional agents that act synergistically with PI3K inhibition.

13. The method of claim 12, wherein an additional agent is a receptor kinase inhibitor, a PI3K pathway inhibitor, or targets the apoptotic machinery.

14. The method of claim 13, wherein the PI3K inhibitor further acts as an allosteric effector of hemoglobin.

15. A compound of the formula (II) 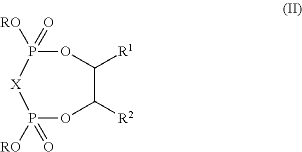
or a pharmaceutically acceptable salt, solvate, and/or prodrug thereof, wherein:
R is hydrogen, a cation, or an organic moiety;
R1 and R2, taken together, may form a ring of 5 or 6 carbon atoms which is optionally substituted, or alternatively, a heterocyclic 5-, 6-, or 7-membered ring, which is optionally substituted; or
R1 and R2, taken together, form a polyol core selected from an aldose, an alditol, and an aldaric acid, which may be substituted; or
one or both of R1 and R2 are carboxy or an ester thereof of from 1 to 10 carbon atoms; or
one of R1 and R2 form a pyrophosphate ring, which may be further substituted by a carboxy group or ester thereof.

16. A method for treating a disease associated with PI3K activity or hypoxia, comprising, administering a compound or pharmaceutical composition of claim 15.

17. The method of claim 16, wherein the disease is cancer, heart failure, anemia, or Alzheimer's disease.

18. The method of claim 17, wherein the condition is a cancer selected from ovarian, cervical, endometrial, colorectal, breast, pancreatic, gastric, glioblastoma, melanoma, liver, prostate, leukemia, lymphoma, head and neck, gastric, kidney, and lung cancer.
